(19) United States
(12) Patent Application Publication
(10) Pub. No.: US 2005/0038239 A1
(43) Pub. Date: Feb. 17, 2005

(54) Novel compositions

(76) Inventor: Ian Catchpole, Stevenage, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/480,424

(22) PCT Filed: Jun. 14, 2002
(86) PCT No.: PCT/GB02/02728

(30) Foreign Application Priority Data
Jun. 15, 2001 (GB) 0114719.8

(51) Int. Cl. C12Q 1/68; C07H 21/04

(52) U.S. Cl. 536/24.3; 530/352; 435/6

(57) ABSTRACT

The present invention relates to compositions comprising DNA attached to one or more functional moities via a locked nucleic acid oligonucleotide. In particular the present invention provides compositions comprising a plasmid containing a gene encoding a protein of interest, wherein said plasmid may be introduced to a tissue or cell and the gene expressed, complexed to the locked nucleic acid functional moiety

The present invention relates to compositions comprising DNA attached to one or more functional moities via a locked nucleic acid oligonucleotide. In particular the present invention provides compositions comprising a plasmid containing a gene encoding a protein of interest, wherein said plasmid may be introduced to a tissue or cell and the gene expressed, complexed to the LNA-functional moiety.

Plasmid based delivery of genes, particularly for immunisation or gene therapy purposes is known. For example, administration of naked DNA by injection into mouse muscle is outline by Vical in International Patent Application WO90/11092.

Johnston et al WO91/07487 describe methods of transferring a gene to vertebrate cells, by the use of microprojectiles that have been coated with a polynucleotide encoding a gene of interest, and accelerating the microparticles such that the microparticles can penetrate the target cell.

DNA vaccines usually consist of a bacterial plasmid vector into which is inserted a strong viral promoter, the gene of interest which encodes for an antigenic peptide and a polyadenylation/transcriptional termination sequences. The gene of interest may encode a full protein or simply an antigenic peptide sequence relating to the pathogen, tumour or other agent which is intended to be protected against. The plasmid can be grown in bacteria, such as for example E. coli and then isolated and prepared in an appropriate medium, depending upon the intended route of administration, before being administered to the host. Following administration the plasmid is taken up by cells of the host where the encoded peptide is produced. The plasmid vector will preferably be made without an origin of replication which is functional in eukaryotic cells, in order to prevent plasmid replication in the mammalian host and integration within chromosomal DNA of the animal concerned.

There are a number of advantages of DNA vaccination relative to traditional vaccination techniques. First, it is predicted that because of the proteins which are encoded by the DNA sequence are synthesised in the host, the structure or conformation of the protein will be similar to the native protein associated with the disease state. It is also likely that DNA vaccination will offer protection against different strains of a virus, by generating cytotoxic T lymphocyte response that recognise epitopes from conserved proteins. Furthermore, because the plasmids are taken up by the host cells where antigenic protein can be produced, a long-lasting immune response will be elicited. The technology also offers the possibility of combing diverse immunogens into a single preparation to facilitate simultaneous immunisation in relation to a number of disease states.

Helpful background information in relation to DNA vaccination is provided in Donnelly et al “DNA vaccines” Ann. Rev Immunol. 1997 15:617-648, the disclosure of which is included herein in its entirety by way of reference.

Despite the numerous advantages associated with DNA vaccination relative to traditional vaccination therapies there is nonetheless a desire to develop improvements which will serve to increase the immune response induced by the protein which is encoded by the plasmid DNA administered to an animal. The present invention addresses these issues.

Locked nucleic acid (LNA) is an analogue of RNA or DNA. The term LNA is used to describe both nucleotide monomers, in which the ribose ring is constrained by a methylene linkage between the 2′-oxygen and the 4′-carbon, and also oligonucleotides that contain one or more monomers of locked nucleic acid. The methylene bridge linkage can be through oxygen, (oxy-LNA), sulphur, (thio-LNA) and amine, (amino-LNA). The confirmation restriction increases binding affinity for complementary sequences (Dwaine A. Braasch and David R. Corey, Chemistry and Biology 8 (2001) 1-7). The introduction of LNA monomers into DNA or RNA oligonucleotides increases affinity for complementary DNA or RNA sequences, ie. measured as thermal stability of duplexes, eg. melting temperature, (Tm), increases in the range of 3-8° C., depending on the actual base, per LNA monomer present in the oligonucleotide., (Dwaine A. Braasch and David R. Corey, Chemistry and Biology 8 (2001) 1-7). Synthesis of LNA is described in International Patent Application No. WO99/14226.

Although triplex formation of LNA oligonucleotides with short double stranded DNA oligonucleotides has been described, (74, 75), no report has yet been published on the properties of LNA oligonucleotides as strand displacement agents in conjunction with large supercoiled plasmid DNA molecules, which in part is the subject matter of the present invention. Indeed, it has been recently suggested that the charged backbone of LNA oligonucleotides would make them less efficient strand displacement agents than uncharged PNA oligonucleotides, (Dwaine A. Braasch and David R. Corey, Chemistry and Biology 8 (2001) 1-7). The present inventors here provide clear evidence that LNA oligonucleotides are at least as efficient strand displacement agents of supercoiled plasmid DNA as their PNA derived counterparts. We also provide strong evidence that once bound to plasmid DNA, LNA oligonucleotides are more stably attached to plasmid DNA than PNA oligonucleotides and can remain bound when exposed to harsh condition whereas PNA oligonucleotides do not. This is advantageous when considering formulating such DNA/LNA complexes for pharmaceutical administration.

Linking peptide and other material (eg. fluorescent labels such as rhodamime) to DNA plasmid by means of a Peptide nucleic acid oligonucleotide is known (U.S. Pat. No. 6,165,720). These have also been used to transfect cells. Although reports have been made of PNA/DNA/PNA triplexes surviving quite harsh conditions (50), such studies were only performed on short DNA oligonucleotides and not upon large supercoiled plasmid DNA, where stability of bound PNA to a range of external conditions has not been reported. Moreover, it has been found by the present inventors that such complexes are not sufficiently stable to enable a PNA-coupled fluorophore or peptide to remain attached to plasmid DNA when administered in a pharmaceutical or vaccine formulation, especially for (particle mediated immunotherapeutic delivery) PMID. Additionally, the inventors have found labelling of plasmid DNA with PNA oligonucleotides to have variable efficiency, poor reproducibility and constraints on reaction conditions in requiring low or no salt and low pH, (<6), for optimal PNA labelling.

The present invention provides LNA—conjugates and binding of these conjugates to plasmid DNA containing a gene under the control of a promoter such that the gene may be expressed in vivo. The LNA conjugate is stable and can be administered in vivo with the plasmid DNA allowing co-localisation of the plasmid and the functional moiety within the cells whilst still retaining the ability of the gene to be expressed. LNA oligonucleotides, advantageously are not subject to degradation by intracellular Dnase enzymes, (Dwaine A. Braasch and David R. Corey, Chemistry and Biology 8 (2001) 1-7).

The LNA conjugate comprises an oligonucleotide of between 7-25, preferably 10-20, more preferably 11-15 bases at least one of which is a locked nucleic acid preferably at least half, more preferably the entire oligonucleotide is made of LNA bases. Typically, at least a sequence of at least 13 LNA residues is preferred for optimal stability, when bound to the plasmid DNA. Preferred LNA molecules for use in any aspect of the present invention are listed in Tables 1 and 3. A particularly preferred LNA oligonucleotide is shown in table 1 as LNA 4. The LNA oligonucleotide should be free from self-complementary base-pairing sequences for optimal binding to DNA. An alternative embodiment can be envisaged where complementary sequences to further LNA oligonucleotides are present in intial bound LNA oligonucleotides such that an array of LNA oligonucleotide can be bound to a single LNA complementary site within DNA, formed by LNA: LNA hybridization between LNA oligonucleotides.

In the present invention the LNA is conjugated to a functional moiety, so that once the LNA is associated with the DNA plasmid encoding a gene of interest and administered to a host, the DNA plasmid can express the gene and allow the function of the attached moiety. Preferably the functional moiety is a biological response modifier, such as an expression enhancer or vaccine adjuvant.

Preferably the functional moiety is selected from the group of organic molecules, proteins, peptide or carbohydrate or lipid moities. In particular: Nuclear localisation peptides, peptides that have the ability to cross the plasma membrane of eukaryotic cells, (“cell penetrating peptides”) endosomal escape peptides; cell targeting and binding peptides or protein. Also proteins or peptides containing transcription activation domains could be conjugated to LNA oligonucleotides for this process. Similarly, molecules having adjuvant or immunostimulatory activity may be attached.

A range of functionally active proteins and peptides could be coupled via LNA oligonucleotides to DNA for a variety of different applications. These can be divided into groups of functional peptides such as those that demonstrate intracellular transport properties including nuclear localisation, cell penetration and endosomal release, and small molecules and proteins that exhibit adjuvant activity. In more detail peptide based nuclear localisation signals, (NLS), are short stretches of amino acids carried within proteins that are localised to the nucleus. These have been somewhat arbitrarily categorised into three classes known as classical, (basically charged stretches), bipartite, (two stretches of basic charge separated by a 10-20 amino acid intervening spacer) and non-classical, (not highly basically charged). The current state of knowledge as to how NLS peptides act to promote cytoplasm to nuclear uptake of proteins has been recently reviewed, (3,4). The presence of a NLS peptide attached to plasmid DNA is designed to improve nuclear uptake across the nuclear membrane of transfected cells, without requiring cell division and also in non-dividing cells. As a result elevated and more rapid gene expression is achieved. An increase in the percentage of cells to which DNA is delivered where the DNA successfully reaches the nucleus and encoded genes are expressed is also achieved. Examples of such nuclear localisation peptides that could be considered for use in this system, although this does not preclude the use of other such peptides, by conjugation to LNA oligonucleotides include: SV40 T Ag NLS and chimeric combinations with other peptides, eg. MPG, (5,6,7, 21), adenovirus fibre peptide, (8), the M9 sequence of hnRNP A1, (9), polyomavirus and SV40 major capsid protein VP1, (10, 23), HIV-1 tat protein, (11), antennapedia peptide and other trojan peptide or penetration based peptide domains including those from nuclear growth factors, (12,13,14, 15), peptides and proteins derived from steroid hormone receptors and their co-factors required for nuclear transport, eg. ARNT derived peptide, (16), nucleoplasmin peptide, (17), Vir D2 peptide, (18), c-myc peptide, (19), polylysine (20), pp65 (UL83) tegument protein of human CMV, (22), hepatitis delta antigen, (23) and peptides based upon the importin beta-binding domain, leucine zipper regions and MADS box regions of nuclear proteins, (25).

Peptides that have the ability to cross the plasma membrane of eukaryotic cells in a relatively energy independent manner have been described as cell-penetrating peptides, (26). These will be useful to facilitate uptake of extracellular plasmid DNA and result in an increase in the percentage of cells where plasmid encoded genes are expressed. Some such peptides also have NLS properties and may have been listed earlier, but others without such NLS activity that should be considered, but such a list is by no means all encompassing, include VP22 from HSV-1 and similar peptides from homologous Herpes virus proteins, (27), HPV-1 type 16 capsid protein L2 and similar peptides from homologous Papillomavirus virus proteins, (28), PEA: the exotoxin A gene from Pseudomonas aeruginosa, (29), the preS2-domain of hepatitis-B virus surface antigens, (30), the signal sequence or membrane translocating sequence (MTS) peptides and synthetic peptides such as galparan or transportan, (26).

A number of peptides have been described that have endosomalytic activity and this is considered to be an important function to incorporate into a synthetic gene delivery system to allow escape of plasmid DNA from the endosome to enable gene expression. Such peptides considered for use in this invention include but are not limited to: the influenza hemagluttinin HA-2 peptide, (31), vesicular stomatitis virus G protein, Alzheimer beta-amyloid peptide, (32), GALA, (33), alpha-helical peptide, (34), KALA, (35), EALA, (36), melittin-derived peptide, (37) and other chemicals and polymers that are thought to promote endosomal release such as chloroquine and PEI, (40).

It is similarly envisaged that the attachment to LNA oligonucleotides of peptides or other moieties that enhance cell targeting and binding when annealed to plasmid DNA enables improvements in cell specific uptake and targeting of DNA expression. Peptides and other cell targeting moieties that can be considered for use with this invention could include, by no means exclusively: monoclonal antibodies directed to cell surface receptors, eg. CD4, (38), peptide or protein ligands for cell surface receptors such as basic fibroblast growth factor, (39), transferrin, (40), Flt-3-ligand, (44), integrin-binding peptides such as those containing the RGD motif, (45), sugars which act as ligands for cell surface receptors such as mannose, (41), or lactosylated or gluconoylated polylysine, (42), or galactose, (43) and folate and other such cell specific ligands described within (46, 47).

Also considered in this invention are chimeric peptides combining the functional activities of nuclear localisation, endosomal escape and cellular targeting for attachment via LNA oligonucleotides to plasmid which enhance gene expression such as those described in (48).

The use of peptides and proteins containing transcription-activating domains are an embodiment of the invention. Conjugated LNA oligonucleotides can be designed to bind sites within a plasmid close to binding sites for RNA polymerase within a promoter required for gene expression from the plasmid, but not such that progression of the enzyme is impeded. Gene expression is expected to be enhanced by potentiated binding of RNA polymerase and its co-factors. There are three classes of major transcription activation domains that have been described and these are: acidic, glutamine-rich and proline-rich, (69, 70, 71, 72). Examples of such domains that could be included in this invention include, although this list is by no means exclusive, include VP16, (amino acids 337-347), Oct-2 (amino acids 143-160), and Sp1 (amino acids 340-385).

Yet another embodiment of the invention is to link immunomodulatory agents, especially immunostimulatory agents, either as protein, peptide, lipid or small molecule chemicals, but not necessarily excluding alternative formulations, via LNA oligonucleotides to plasmid DNA. This offers advantages to delivery systems where it would become possible to co-deliver immune adjuvants and DNA in one formulation, even to the same cell. The invention allows to co-deliver smaller, more specifically targeted doses of adjuvants with DNA, and reduce some of the problems from large systemic doses of immunomodulatory agents. This could be especially advantageous for PMID. Potential immunostimulatory agents include, but this list is by no means exhaustive and does not preclude other agents: synthetic imidazoquinolines such as imiquimod [S-26308, R-837], (58) and resiquimod [S-28463, R-848] (59), Schiff bases of carbonyls and amines that are constitutively expressed on antigen presenting cell and T-cell surfaces, such as tucaresol (60), cytokine, chemokine and co-stimulatory molecules as either protein or peptide, this would include pro-inflammatory cytokines such as GM-CSF, IL-1 alpha, IL-1 beta, TGF-alpha and TGF-beta, Th1 inducers such as interferon gamma, IL-2, IL-12, IL-15 and IL-18, Th2 inducers such as IL-4, IL-5, IL-6, IL-10 and IL-13 and other chemokine and co-stimulatory genes such as MCP-1, MIP-1 alpha, MIP-1 beta, RANTES, TCA-3, CD80, CD86 and CD40L, (63), other immunostimulatory targeting ligands such as CTLA-4 and L-selectin, (63), apoptosis stimulating proteins and peptides such as Fas, (49), synthetic lipid based adjuvants, such as vaxfectin, (64), squalene, alpha-tocopherol, polysorbate 80, DOPC and cholesterol, endotoxin, [LPS], (67), and other potential ligands that trigger Toll receptors to produce Th1-inducing cytokines, such as synthetic Mycobacterial lipoproteins, Mycobacterial protein p19, peptidoglycan, teichoic acid and lipid A, (68).

Certain preferred adjuvants for eliciting a predominantly Th1-type response include, for example, a Lipid A derivative such as monophosphoryl lipid A, or preferably 3-de-O-acylated monophosphoryl lipid A. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins.

Particularly preferred adjuvants for linking to DNA plasmids via the LNA are CpG oligo- and di-nucleotides, (65, 66). Accordingly there is provided a novel oligonucleotide composition comprising a first region having an oligonucleotide sequence comprising at least one LNA and a second region having an immunostimulatory oligonucleotide region containing at least one CG unmethylated di-nucleotide motif. The immunostimulatory sequence is often: Purine, Purine, C, G, pyrimidine, pyrimidine; wherein the dinucleotide CG motif is not methylated. The preferred oligonucleotides for use in adjuvants or vaccines of the present invention preferably contain two or more dinucleotide CpG motifs separated by at least three, more preferably at least six or more nucleotides. The oligonucleotides of the present invention are typically deoxynucleotides. In a preferred embodiment the internucleotide in the oligonucleotide is phosphorodithioate, or more preferably a phosphorothioate bond, although phosphodiester and other internucleotide bonds are within the scope of the invention including oligonucleotides with mixed internucleotide linkages. Methods for producing phosphorothioate oligonucleotides or phosphorodithioate are described in U.S. Pat. No. 5,666,153, U.S. Pat. No. 5,278,302 and WO95/26204. Examples of preferred oligonucleotides have the following sequences. The sequences preferably contain phosphorothioate modified internucleotide linkages.

OLIGO 1 (SEQ ID NO: 1):
TCC ATG ACG TTC CTG ACG TT (CpG 1826)
OLIGO 2 (SEQ ID NO: 2):
TCT CCC AGC GTG CGC CAT (CpG 1758)
OLIGO 3 (SEQ ID NO: 3):
ACC GAT GAC GTC GCC GGT GAC GGC ACC ACG
OLIGO 4 (SEQ ID NO: 4):
TCG TCG TTT TGT CGT TTT GTC GTT (CpG 2006)
OLIGO 5 (SEQ ID NO: 5):
TCC ATG ACG TTC CTG ATG CT (CpG 1668)

Alternative CpG oligonucleotides may comprise the preferred sequences above in that they have inconsequential deletions or additions thereto. The CpG oligonucleotides utilised in the present invention may be synthesised by any method known in the art (e.g. EP 468520). Conveniently, such oligonucleotides may be synthesised utilising an automated synthesiser.

The oligonucleotides utilised in the present invention are typically deoxynucleotides. In a preferred embodiment the internucleotide bond in the oligonucleotide is phosphorodithioate, or more preferably phosphorothioate bond, although phosphodiesters are within the scope of the present invention. Oligonucleotide comprising different internucleotide linkages are contemplated, e.g. mixed phosphorothioate phosphodiesters. Other internucleotide bonds which stabilise the oligonucleotide may be used.

Fluorescently labelled LNA oligonucleotides are used to illustrate the present invention and are available from commercial sources: Proligo LLC, Boulder, Colo., USA. Other modifications of LNA oligonucleotides can be provided by, for example, aqueous Diels-Alder bioconjugation reactions to produce LNA oligonucleotides modified by, for example, biotin, PEG conjugates and maleimide. Standard methods can also be used for the attachment of reactive and protected reactive groups such as primary amino groups, sulphydryl groups and trityl protected sulphydryl groups to LNA oligonucleotides. Fluorescent labels and other small molecules can be attached to such chemically modified LNA oligonucleotides in several standard ways using commercially available reagents and labelling kits including, for example, fluorescently labelled streptavidins, (Perbio Science AB: Pierce Chemical Co. Rockford, Ill., USA), which can be simply and specifically bound to biotinylated LNA oligonucleotides, or the Alexa Fluor labelling kits, (Molecular Probes Inc., Eugene, Oreg., USA), which can simply be reacted with primary amine modified LNA oligonucleotides or LNA oligonucleotide conjugates with peptides containing primary amine groups.

Peptides and proteins may be attached to modified LNA oligonucleotides by using a range of commercially available cross-linking reagents allowing coupling to reactive chemical groups that are either naturally occurring in proteins or can be simply incorporated into commercially available synthetic peptides. A range of potential peptide linkages to LNA oligonucleotides is exemplified below:
i) Peptides synthesised with C-terminal sulphydryl groups can be simply coupled to streptavidin or neutravidin, eg. commercially available EZ-link Maleimide Activated Neutravidin Biotin Binding Protein, (Perbio Science AB: Pierce Chemical Co. Rockford, Ill., USA), and simply bound to biotinylated LNA oligonucleotides,
ii) Peptides synthesised with C-terminal sulphydryl groups can be directly coupled to modified maleimide labelled LNA oligonucleotides,
iii) Peptides synthesised with C-terminal sulphydryl groups can be coupled to the heterobifunctional cross-linker SPDP, (N-Succinimidyl 3-[2-pyridyldithio]propionate), which is commercially available, (Perbio Science AB: Pierce Chemical Co. Rockford, Ill., USA), and this can in turn be coupled to LNA oligonucleotides modified to contain a primary amine group, (1).
   The examples described above are by no means exhaustive and other potential coupling methods that have been considered include utilisation of the amino, aryl, carboxyl and hydroxyl groups found on peptides or proteins and have been extensively reviewed, (2). Other heterobifunctional cross-linking reagents are available for coupling such reactive groups including carbodiimide cross-linkers to couple carboxyl groups to amines, eg. 1-ethyl-3-(3-dimethylaminopropyl)-carbodiimide hydrochloride and other cross-linking reagents that couple to sulphydryl groups, (eg. haloacetyls or pyridyl disuphide), or amino groups, eg. imidoesters or N-hydrosuccinimide-esters including succimidyl 4-(N-maleimidomethyl) cyclohexane-l-carboxylate (SMCC) and succimidyl-4-(p-maleimidophenyl)-butyrate (SMPB).

One preferred embodiment of this invention is to design the linkage of the functional moiety to the LNA oligonucleotide such that it can be selectively cleaved, (perhaps in order to exert a biological response), from the LNA oligonucleotide and bound plasmid once they have been delivered to a cell. One such example of this is described in Example 9 where a CpG adjuvant, as a phosphorothioate oligonucleotide, is linked to an LNA oligonucleotide by a single DNA phosphoramidate residue, which leaves the ‘hybrid’ oligonucleotide available for cleavage by cellular phosphodiester ezymes upon delivery to the endosomal comparment of the cell. Cleavage could then release the CpG adjuvant as a free phosphorothioate oligonucleotide to exert its biological effect.

In a preferred embodiment of the present invention the LNA—conjugate is associated with a DNA molecule encoding a gene, said DNA molecule having a sequence complementary to the LNA oligonucleotide. The DNA is preferably in the form of a plasmid and preferably encodes an antigen or therapeutic protein.

The plasmid is preferably without a functional origin of replication in order to prevent plasmid replication in the host to which it is administered. The promoter is preferably a strong viral promoter such as a CMV promoter.

The plasmid can be provided with a plurality of LNA complementary binding sequences to enable a plurality of LNA/conjugates to bind. The conjugates may have discrete different functional moities. Thus in one aspect of the invention the plasmid may bind to an LNA linked to a nuclear localisation peptide and an LNA linked to a small molecule adjuvant. Typically the plasmid will be provided with 4 or more complementary LNA binding sequences preferably 10 to 20 sequences, but up to 100 sequences are possible. Accordingly in one aspect of the invention there is provided a plasmid LNA conjugate complex wherein there is at least four LNA conjugates bound to the plasmid.

In a preferred embodiment the antigen is capable of eliciting an immune response against a human pathogen, which antigen or antigenic composition is derived from HIV-1, (such as tat, nef, gp120 or gp160, gp40, p24, gag, env, vif, vpr, vpu, rev), human herpes viruses, such as gH, gL gM gB gC gK gE or gD or derivatives thereof or Immediate Early protein such as ICP27, ICP47, IC P4, ICP36 from HSV1 or HSV2, cytomegalovirus, especially Human, (such as gB or derivatives thereof), Epstein Barr virus (such as gp350 or derivatives thereof), Varicella Zoster Virus (such as gpI, II, III and IE63), or from a hepatitis virus such as hepatitis B virus (for example Hepatitis B Surface antigen or Hepatitis core antigen or pol), hepatitis C virus antigen and hepatitis E virus antigen, or from other viral pathogens, such as paramyxoviruses: Respiratory Syncytial virus (such as F and G proteins or derivatives thereof), or antigens from parainfluenza virus, measles virus, mumps virus, human papilloma viruses (for example HPV6, 11, 16, 18, eg L1, L2, E1, E2, E3, E4, E5, E6, E7), flaviviruses (e.g. Yellow Fever Virus, Dengue Virus, Tick-borne encephalitis virus, Japanese Encephalitis Virus) or Influenza virus cells, such as HA, NP, NA, or M proteins, or combinations thereof), or antigens derived from bacterial pathogens such as Neisseria spp, including N. gonorrhea and N. meningitidis, eg, transferrin-binding proteins, lactoferrin binding proteins, PilC, adhesins); S. pyogenes (for example M proteins or fragments thereof, C5A protease, S. agalactiae, S. mutans; H. ducreyi; Moraxella spp, including M catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M tuberculosis (for example ESAT6, Antigen 85A, -B or -C, MPT44, MPT59, MPT45, HSP10, HSP65, HSP70, HSP 75, HSP90, PPD 19kDa [Rv3763], PPD 38kDa [Rv0934]), M bovis, M leprae, M avium, M. paratuberculosis, M smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli enteropathogenic E. coli (for example shiga toxin-like toxin or derivatives thereof); Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y pestis, Y pseudotuberculosis; Campylobacter spp, including C. jejuni (for example toxins, adhesins and invasins) and C. coli; Salmonella spp, including S. typhi, S. paratyphi S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C tetani (for example tetanus toxin and derivative thereof), C botulinum (for example botulinum toxin and derivative thereof), C difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or derived from parasites such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans.

Other preferred specific antigens for M. tuberculosis are for example Rv2557, Rv2558, RPFs: Rv0837c, Rv1884c, Rv2389c, Rv2450, Rv1009, aceA (Rv0467), PstS1, (Rv0932), SodA (Rv3846), Rv2031c 16kDal ., Tb Ra12, Tb H9, Tb Ra35, Tb38-1, Erd 14, DPV, MTI, MSL, mTTC2 and hTCC1 (WO 99/51748). Proteins for M. tuberculosis also include fusion proteins and variants thereof where at least two, preferably three polypeptides of M. tuberculosis are fused into a larger protein. Preferred fusions include Ra12-TbH9-Ra35, Erd14-DPV-MTI, DPV-MTI-MSL, Erd14-DPV-MTI-MSL-mTCC2, Erd14-DPV-MTI-MSL, DPV-MTI-MSL-mTCC2, TbH9-DPV-MTI (WO 99/51748).

Most preferred antigens for Chlamydia include for example the High Molecular Weight Protein (HWMP) (WO 99/17741), ORF3 (EP 366 412), and putative membrane proteins (Pmps). Other Chlamydia antigens of the vaccine formulation can be selected from the group described in WO 99/28475.

Preferred bacterial vaccines comprise antigens derived from Streptococcus spp, including S. pneumoniae (PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Other preferred bacterial vaccines comprise antigens derived from Haentophilus spp., including H. influenzae type B (for example PRP and conjugates thereof), non typeable H. influenzae, for example OMP26, high molecular weight adhesins, P5, P6, protein D and lipoprotein D, and fimbrin and fimbrin derived peptides (U.S. Pat. No. 5,843,464) or multiple copy varients or fusion proteins thereof.

The antigens that may be used in the present invention may further comprise antigens derived from parasites that cause Malaria For example, preferred antigens from Plasmodia falciparum include RTS,S and TRAP. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. It's full structure is disclosed in the International Patent Application No. PCT/EP92/02591, published under Number WO 93/10152 claiming priority from UK patent application No.9124390.7. When expressed in yeast RTS is produced as a lipoprotein particle, and when it is co-expressed with the S antigen from HBV it produces a mixed particle known as RTS,S. TRAP antigens are described in the International Patent Application No. PCT/GB89/00895, published under WO 90/01496. A preferred embodiment of the present invention is a Malaria vaccine wherein the antigenic preparation comprises a combination of the RTS, S and TRAP antigens. Other plasmodia antigens that are likely candidates to be components of a multistage Malaria vaccine are P. faciparum MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp.

The invention contemplates the use of an anti-tumour antigen and be useful for the immunotherapeutic treatment of cancers. For example, tumour rejection antigens such as those for prostrate, breast, colorectal, lung, pancreatic, renal or melanoma cancers. Exemplary antigens include MAGE 1, 3 and MAGE 4 or other MAGE antigens such as disclosed in WO99/40188, PRAME, BAGE, Lage (also known as NY Eos 1) SAGE and HAGE (WO 99/53061) or GAGE (Robbins and Kawakami, 1996, Current Opinions in Immunology 8, pps 628-636; Van den Eynde et al., International Journal of Clinical & Laboratory Research (submitted 1997); Correale et al. (1997), Journal of the National Cancer Institute 89, p293. Indeed these antigens are expressed in a wide range of tumour types such as melanoma, lung carcinoma, sarcoma and bladder carcinoma.

MAGE antigens for use in the present invention may be expressed as a fusion protein with an expression enhancer or an Immunological fusion partner. In particular, the Mage protein may be fused to Protein D from Heamophilus influenzae B. In particular, the fusion partner may comprise the first ⅓ of Protein D. Such constructs are disclosed in Wo99/40188. Other examples of fusion proteins that may contain cancer specific epitopes include bcr/abl fusion proteins.

In a preferred embodiment prostate antigens are utilised, such as Prostate specific antigen (PSA), PAP, PSCA (PNAS 95(4) 1735-1740 1998), PSMA or antigen known as Prostase.

Prostase is a prostate-specific serine protease (trypsin-like), 254 amino acid-long, with a conserved serine protease catalytic triad H-D-S and a amino-terminal pre-propeptide sequence, indicating a potential secretory function (P. Nelson, Lu Gan, C. Ferguson, P. Moss, R. Gelinas, L. Hood & K. Wand, “Molecular cloning and characterisation of prostase, an androgen-regulated serine protease with prostate restricted expression, In Proc. Natl. Acad. Sci. USA (1999) 96, 3114-3119). A putative glycosylation site has been described. The predicted structure is very similar to other known serine proteases, showing that the mature polypeptide folds into a single domain. The mature protein is 224 amino acids-long, with one A2 epitope shown to be naturally processed.

Prostase nucleotide sequence and deduced polypeptide sequence and homologs are disclosed in Ferguson, et al. (Proc. Natl. Acad. Sci. USA 1999, 96, 3114-3119) and in International Patent Applications No. WO 98/12302 (and also the corresponding granted patent U.S. Pat. No. 5,955,306), WO 98/20117 (and also the corresponding granted patents U.S. Pat. No. 5,840,871 and U.S. Pat. No. 5,786,148) (prostate-specific kallikrein) and WO 00/04149 (P703P).

The present invention provides antigens comprising prostase protein fusions based on prostase protein and fragments and homologues thereof (“derivatives”). Such derivatives are suitable for use in therapeutic vaccine formulations which are suitable for the treatment of a prostate tumours. Typically the fragment will contain at least 20, preferably 50, more preferably 100 contiguous amino acids as disclosed in the above referenced patent and patent applications.

A further preferred prostate antigen is known as P501S, sequence ID no 113 of WO98/37814. Immunogenic fragments and portions encoded by the gene thereof comprising at least 20, preferably 50, more preferably 100 contiguous amino acids as disclosed in the above referenced patent application, are contemplated. A particular fragment is PS108 (WO 98/50567).

Other prostate specific antigens are known from W098/37418, and WO/004149. Another is STEAP PNAS 96 14523 14528 7-12 1999.

Other tumour associated antigens useful in the context of the present invention include: Plu-1 J Biol. Chem 274 (22) 15633-15645, 1999, HASH-1, HasH-2, Cripto (Salomon et al Bioessays 199, 21 61-70, U.S. Pat. No. 5,654,140) Criptin U.S. Pat. No. 5,981,215,. Additionally, antigens particularly relevant for vaccines in the therapy of cancer also comprise tyrosinase and survivin.

The present invention is also useful in combination with breast cancer antigens such as Muc-1, Muc-2, EpCAM, her 2/Neu, mammaglobin (U.S. Pat. No. 5,668,267) or those disclosed in WO/00 52165, WO99/33869, WO99/19479, WO 98/45328. Her 2 neu antigens are disclosed inter alia, in U.S. Pat. No. 5,801,005. Preferably the Her 2 neu comprises the entire extracellular domain (comprising approximately amino acid 1-645) or fragments thereof and at least an immunogenic portion of or the entire intracellular domain approximately the C terminal 580 amino acids. In particular, the intracellular portion should comprise the phosphorylation domain or fragments thereof. Such constructs are disclosed in WO00/44899. A particularly preferred construct is known as ECD PD a second is known as ECD □PD. (See WO/00/44899.)

The her 2 neu as used herein can be derived from rat, mouse or human.

The plasmid may encode antigens associated with tumour-support mechanisms (e.g. angiogenesis, tumour invasion) for example tie 2, VEGF.

Vaccines of the present invention may also be used for the prophylaxis or therapy of chronic disorders in addition to allergy, cancer or infectious diseases. Such chronic disorders are diseases such as asthma, atherosclerosis, and Alzheimers and other auto-immune disorders. Vaccines for use as a contraceptive may also be considered.

Antigens relevant for the prophylaxis and the therapy of patients susceptible to or suffering from Alzheimer neurodegenerative disease are, in particular, the N terminal 39-43 amino acid fragment (A□the amyloid precursor protein and smaller fragments. This antigen is disclosed in the International Patent Application No. WO 99/27944-(Athena Neurosciences).

Potential self-antigens that could be included as vaccines for auto-immune disorders or as a contraceptive vaccine include: cytokines, hormones, growth factors or extracellular proteins, more preferably a 4-helical cytokine, most preferably IL13. Cytokines include, for example, IL1, IL2, IL3, IL4, IL5, IL6, IL7, IL8, IL9, IL10, IL11, IL12, IL13, IL14, IL15, IL16, IL17, IL18, IL20, IL21, TNF, TGF, GMCSF, MCSF and OSM. 4-helical cytokines include IL2, IL3, IL4, IL5, IL13, GMCSF and MCSF. Hormones include, for example, luteinising hormone (LH), follicle stimulating hormone (FSH), chorionic gonadotropin (CG), VGF, GHrelin, agouti, agouti related protein and neuropeptide Y. Growth factors include, for example, VEGF.

The vaccines of the present invention are particularly suited for the immunotherapeutic treatment of diseases, such as chronic conditions and cancers, but also for the therapy of persistent infections. Accordingly the vaccines of the present invention are particularly suitable for the immunotherapy of infectious diseases, such as Tuberculosis (TB), AIDS and Hepatitis B (HepB) virus infections.

Accordingly there is provided vaccines comprising the present invention for the immunotherapy of infectious diseases such as TB, AIDS and HepB; and their use in the manufacture of medicaments for the immunotherapy of infectious diseases such as TB, AIDS and HepB. In the context of TB, there is provided a method of treating an individual suffering from TB infection, comprising the administration of a vaccine of the present invention to the individual, thereby reducing the bacterial load of that individual. The reduction of bacterial load, consisting of a reduction of the amount of TB found in the lung sputum, leading to the amelioration or cure of the TB disease.

Also, in the context of AIDS, there is provided a method of treatment of an individual susceptible to or suffering from AIDS. The method comprising the administration of a vaccine of the present invention to the individual, thereby reducing the amount of CD4+T-cell decline caused by subsequent HIV infection, or slowing or halting the CD4+T-cell decline in an individual already infected with HIV.

Additionally, in the context of persistant Hepatitis B virus infection, there is provided a method of treatment of an individual susceptible to or suffering from HepB infection. Accordingly, there is provided a method comprising the administration of a vaccine of the present invention to the individual, thereby reducing the level of HepB load in the serum (as measured by DNA clearance) and also reducing the amount of liver damage (as detected by the reduction or stabilisation of serum levels of the enzyme Alanine Transferase (ALT)).

The LNA-conjugate/DNA complex may thus be formulated into a pharmaceutical or immunogenic composition or vaccine. In an embodiment of the invention, a polynucleotide is administered/delivered as “naked” DNA, for example as described in Ulmer et al., Science 259:1745-1749, 1993 and reviewed by Cohen, Science 259:1691-1692, 1993. Here the DNA is formulated in a buffered saline solution and injected directly into tissue. The uptake of naked DNA may be increased by coating the DNA onto biodegradable beads, which are efficiently transported into the cells or by using other well known transfection facilitating agents. LNA-conjugate/DNA may be administered in conjunction with a carrier such as, for example, liposomes. Typically such liposomes are cationic, for example imidazolium derivatives (WO95/14380), guanidine derivatives (WO95/14381), phosphatidyl choline derivatives (WO95/35301), piperazine derivatives (WO95/14651) and biguanide derivatives. The LNA-conjugate/DNA complex may deliver a gene of interest such as CTFR or erythropoetin gene operatively linked to a promoter sequence. Thus a method of correcting or compensating for a disease or disorder whose etiology is characterised by a genetic aberration (such as cystic fibrosis) is provided, which method comprises the step of administrating to a mammalian patient in clinical need thereof a therapeutically effective amount of the construct, preferably incorporated into a carrier.

In still another embodiment, a composition of the present invention can be delivered via a particle bombardment approach, many of which have been described (WO 91/07487). In one illustrative example, gas-driven particle acceleration can be achieved with devices such as those manufactured by Powdeiject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide, are accelerated to high speed within a helium gas jet generated by a hand held device, propelling the particles into a target tissue of interest, typically the skin. The particles are preferably gold beads of a 0.4-4.0 um, more preferably 0.6-2.0 um diameter and the DNA conjugate coated onto these and then encased in a cartridge for placing into the “gene gun”.

Accordingly, there is provided a DNA delivery device comprising dense microbeads coated with DNA plasmid encoding a gene of interest, which plasmid is associated with one or more LNA linked to the functional moiety. Preferably there is provided a vaccine or immunogenic composition functional moiety-LNA-plasmid adsorbed gold microbeads.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

The invention is illustrated by, but not limited to, the following examples.

### EXAMPLE 1

### Comparative Binding of LNA and PNA Oligonucleotides to Supercoiled Plasmid DNA

Plasmids.

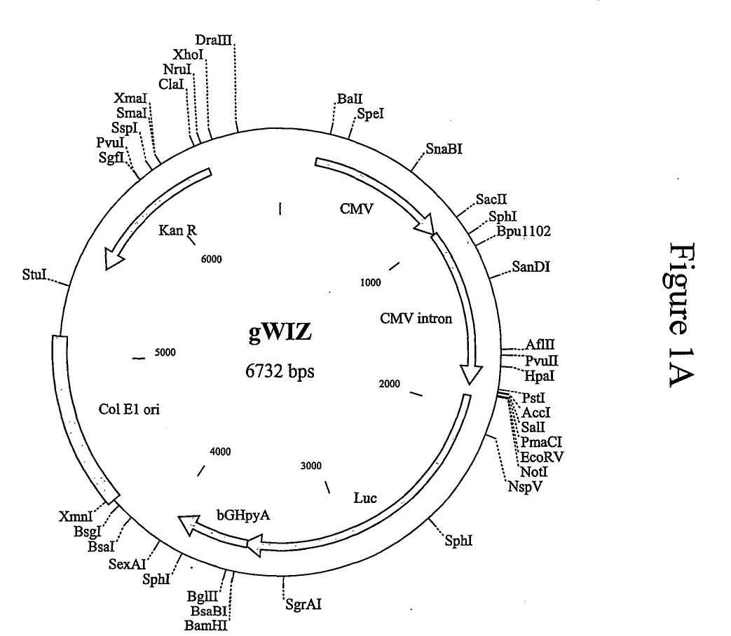

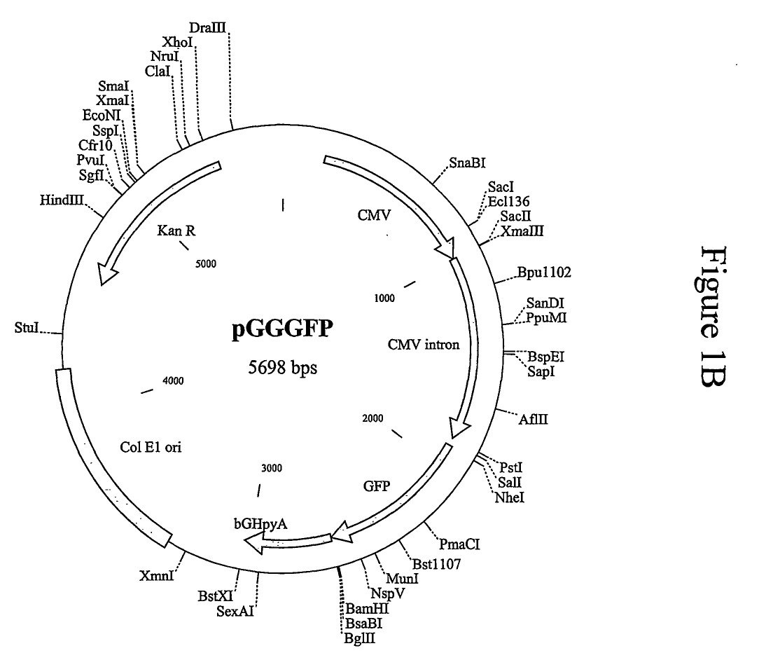

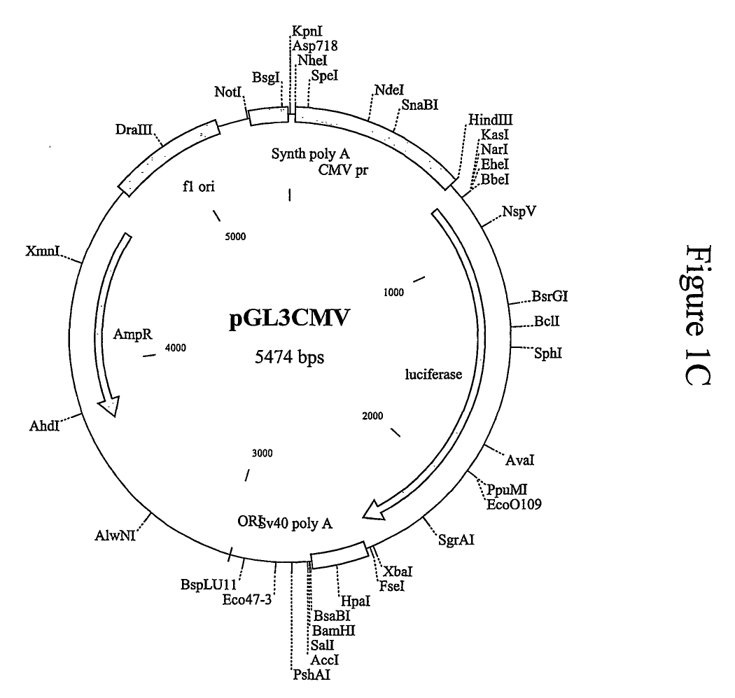

The plasmids used in this study are all described in FIG. 1. Plasmid DNA was prepared by a standard alkaline lysis procedure using the Qiagen MaxiPrep procedure, (Qiagen GmbH, Hilden, Germany), and resuspended in TE, (10 mM TrisHCl, 1 mM EDTA), pH 8.0 at 1 ug/ul. Plasmids were determined as>95% supercoiled upon analysis by agarose gel electrophoresis, (51).

LNA Oligonucleotides.

The LNA oligonucleotides used in this study, (Table 1), were synthesized with rhodamine attached at the 5′ end, (Proligo LLC, Colorado, USA). LNA 5 was made with 50% LNA and 50% DNA monomer residues as this might be expected to have intermediate hybridization properties compared to 100% LNA or 100% DNA oligonucleotides.

TABLE 1

The LNA and PNA
oligonucleotides used in this study

| | No. of | |
|---|---|---|
| | binding | |
| Name | sites. | Sequence |
| LNA2 | 8 | 5′-TAMRA-CTCTCTCTC-3′ |
| LNA4 | 6 | 5′-TAMRA-CTCTCTCTCTCTC-3′ |
| LNA5 | 4 | 5′-TAMRA-CtCtCtCtCtCtCtCtC-3′ |
| GTS PNA | 9-10 | 5′-RhO-O-O-TCTCTCTC-O-O-O-JTJT |
| | | JTJT-CONH2 |
| OsPNA2 | 6 | 5′-ROX-O-O-glyCTCTCTCTCTCTC-O- |
| | | CTCTCTCTCTCTC lys |

LNA residues are displayed in bold upper case, DNA residues are shown in bold lower case, PNA and amino acid residues are shown in italics.
O=8-amino-3,6-dioxaoctanoic acid linker, J=pseudoisocytosine, gly=glycine, lys=lysine.
Number of binding sites refers to the maximum number of theoretical oligonucleotide binding sites present on the gWiz plasmid.
PNA Oligonucleotides.

The PNA oligonucleotides used in this study, (Table 1), were obtained from two sources. The GTS PNA was purchased from GTS Inc., (San Diego, Calif., USA), as the 5′ rhodamine-labelled PNA clamp from a commercially available plasmid labelling system. The OsPNA2 was purchased from Oswel DNA Service, Southampton, UK, as a 5′ rhodamine labelled ‘bis’ PNA or PNA clamp, (52, 54), modified with a 5′/N-terminal glycine residue and a 3′/C-terminal lysine residue. This is thought to improve the stability of ‘bis’ PNAs when bound to DNA, (53). The principle behind the design structure of both PNAs is that they are able to bind to DNA both by standard Watson and Crick base pairing and also by Hoogsten base pairing to form triplex base-paired structures on one strand of double stranded DNA, (50, 52). The ‘O’ residues, (Table 1), separate the PNAs into two regions, the region 3′ or C-terminal to the ‘O’ residue can Hoogsten base pair with DNA and this is optimal for ‘bis’ PNAs or PNA clamps containing J, (pseudocytosine), residues for base pairing at high pH>5-6, whereas C, (cytosine) residues can still show Hoogsten base pairing at low pH<6 (52).

Both LNA and PNA oligonucleotides should bind specifically to multiple homopurine AG binding sites present in the gWiz plasmid, (Table 1, FIG. 1, 50).

FIG. 1 shows the plasmids used in this study
(A) gWiz is a luciferase expression vector, (Gene Therapy Systems [GTS], Inc., San Diego, Calif., USA), this contains multiple AG motifs within the DNA sequence of the bGHpyA region of the plasmid, (GTS Catalogue, 1999) Luc=luciferase, bGHpyA=bovine Growth Hormone polyadenylation region.
(B) pGGGFP is a GFP expression vector, (Gene Therapy Systems [GTS], Inc., San Diego, Calif., USA), this contains multiple AG motifs within the DNA sequence of the bGHpyA region of the plasmid, (50).
(C) pGL3CMV is a luciferase expression vector based upon pGL3 Basic, (Promega Corporation., Madison, Wis., USA), where the CMV immediate early promoter drives luciferase expression, (I. Catchpole, unpublished).
   Incubation of LNA and PNA Oligonucleotides with Supercoiled Plasmid DNA.

Annealing/labelling conditions for PNA oligonucleotides were based upon those described in the literature, (55). In order to maximise the efficiency of PNA binding to supercoiled plasmid DNA labelling was performed in a buffer containing no salt at low pH<6, (10 mM phosphate buffer, 1 mM EDTA, pH 5.8) for 16 hours at 37° C. The low pH should enable cytosine residues to Hoogsten base pair with similar efficiency to pseudocytosine residues, (52), Initially, 10 ug of plasmid DNA was labelled in a total volume of 20 ul, where PNA oligonucleotides were present at a 20× molar excess over the maximum number of potential binding sites present in the plasmid DNA, 10 sites, (see Table 1, 50). For OsPNA2, labelling was performed by heating at 95° C. for 10 minutes followed by 10 minutes at room temperature, (20° C.), following by 16 hours at 37° C., (7).

Plasmid labelling with LNA oligonucleotides was initially performed under comparable conditions to that for PNA labelling, but at pH 7.0, (ie. 10 mM phosphate buffer, 1 mM EDTA, pH 7.0 for 16 hours at 37° C.), with a twenty fold molar excess of oligonucleotide over the maximum number of PNA/LNA binding sites in the plasmid, (ten), for 10 ug of plasmid DNA present in a 20 ul total volume.

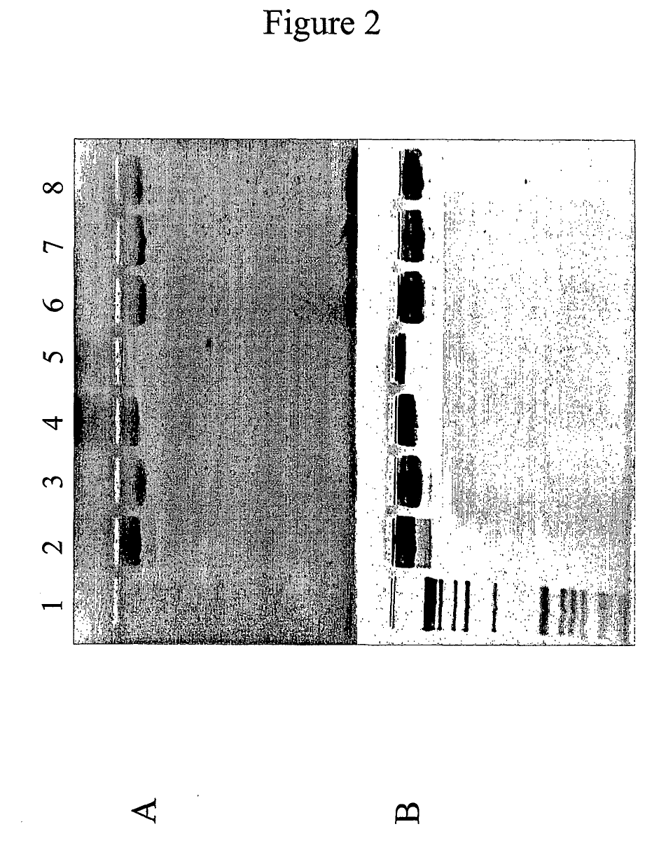

Fractions of the labelling reactions containing 2.5 ug of plasmid DNA were analysed after electrophoresis on 2% agarose/TAE gels, (Sigma, 51), in the absence of ethidium bromide, (EtBr). The presence of rhodamine labelled, (LNA/PNA bound), plasmid DNA was visualised by fluorescence under uv light, (FIG. 2A). Total plasmid was localised by EtBr gel staining, (0.5 ug/ml for 10 mins.), and visualised under uv light, (FIG. 2B), to allow comparison of efficiency of rhodamine and therefore PNA/LNA labelling.

FIG. 2 shows 2% agarose gel electrophoresis, in the absence of ethidium bromide, (EtBr), for analysis of supercoiled plasmid DNAs incubated with rhodamine-labelled LNA or PNA oligonucleotides.
(A) Gel analysis on Multi Analyst package on Gel Doc 1000 system, (Bio-Rad Laboratories, California, USA) before EtBr staining,
(B) As (A) after EtBr staining.
1) 1 ug of 1 kb DNA ladder, (Life Technologies Ltd, Paisley, UK)
2) 2.5 ug pGGGFP plasmid commercially labelled with GTS PNA
3) 2.5 ug gWiz plasmid labelled with GTS PNA rhodamnine labelling kit
4) 2.5 ug gWiz plasmid labelled with rhodamine OsPNA2 at 37° C.
5) 2.5 ug gWiz plasmid labelled with rhodamine OsPNA2 at 95° C./room temperature.
6) 2.5 ug gWiz plasmid labelled with rhodamine LNA2 at 37° C.
7) 2.5 ug gWiz plasmid labelled with rhodamine LNA4 at 37° C.
8) 2.5 ug gWiz plasmid labelled with rhodamine LNA5 at 37° C.
   It is apparent form the data shown in FIG. 2 that all three LNA oligonucleotides used in this study, (LNAs 2, 4 & 5), seemed capable of strand displacement and binding to plasmid DNA, under the conditions described, with at least a similar degree of efficiency to that shown by PNA oligonucleotides.
   Larger Scale Annealing of LNA and PNA Oligonucleotides with Supercoiled Plasmid DNA.

Plasmid labelling reactions were repeated for samples of90-110 ug of plasmid DNA, under similar labelling conditions to those described above, in a volume of 200-500 ul. The ratio of LNA oligonucleotide to plasmid DNA was reduced in this experiment to only a two fold molar excess over a theoretical 10 binding sites per plasmid molecule, whereas the molar excess of OsPNA2 was increased to one hundred fold.

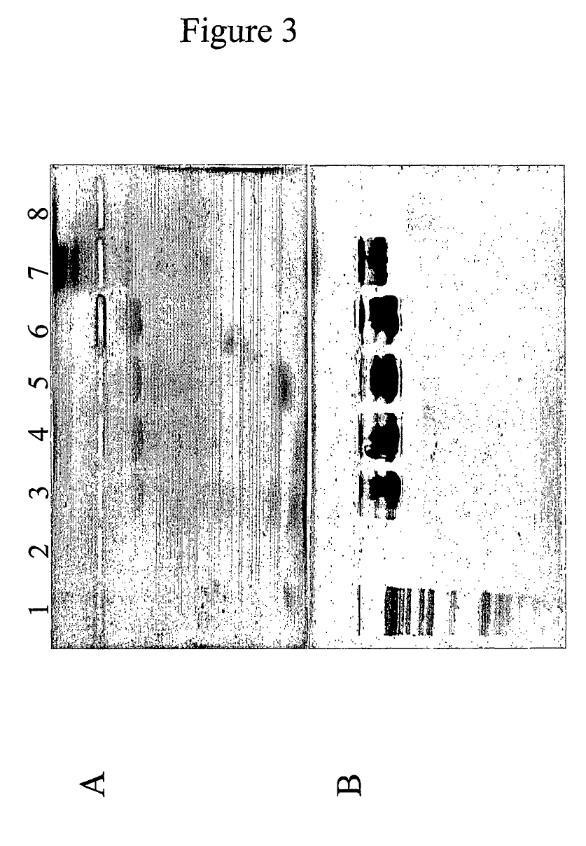

Aliquots of the labelling reactions containing 2.5 ug of plasmid DNA were analysed after electrophoresis on 2% agarose/TAE gels, as described above, (FIG. 3).

FIG. 3 shows 2% agarose gel electrophoresis, in the absence of ethidium bromide, (EtBr), for analysis of supercoiled plasmid DNAs incubated with rhodamine-labelled LNA or PNA oligonucleotides.
(A) Gel analysis on Multi Analyst package on Gel Doc 1000 system, (Bio-Rad Laboratories, California, USA) before EtBr staining,
(B) As (A) after EtBr staining.
1) 1 ug of 1 kb DNA ladder, (Life Technologies Ltd, Paisley, UK)
2) Empty.
3) 2.5 ug gWiz plasmid labelled with rhodamine LNA2 at 37° C.
4) 2.5 ug gWiz plasmid labelled with rhodamine LNA4 at 37° C.
5) 2.5 ug gWiz plasmid labelled with rhodamine LNA5 at 37° C.
6) 2.5 ug gWiz plasmid labelled with GTS PNA rhodamine labelling kit
7) 2.5 ug gWiz plasmid labelled with rhodamine OsPNA2 at 37° C.
8) Empty.

Despite the large differences in molar excess of the LNA oligonucleotides compared to that of OsPNA2, all three LNA oligonucleotides, (LNAs 2, 4 & 5), seemed to have labelled plasmid DNA more efficiently than OsPNA2 as judged by rhodamine fluorescence under uv light, (FIG. 3).

Verification of Oligonucleotide Binding to Cognate Binding Sites within Plasmid DNA.

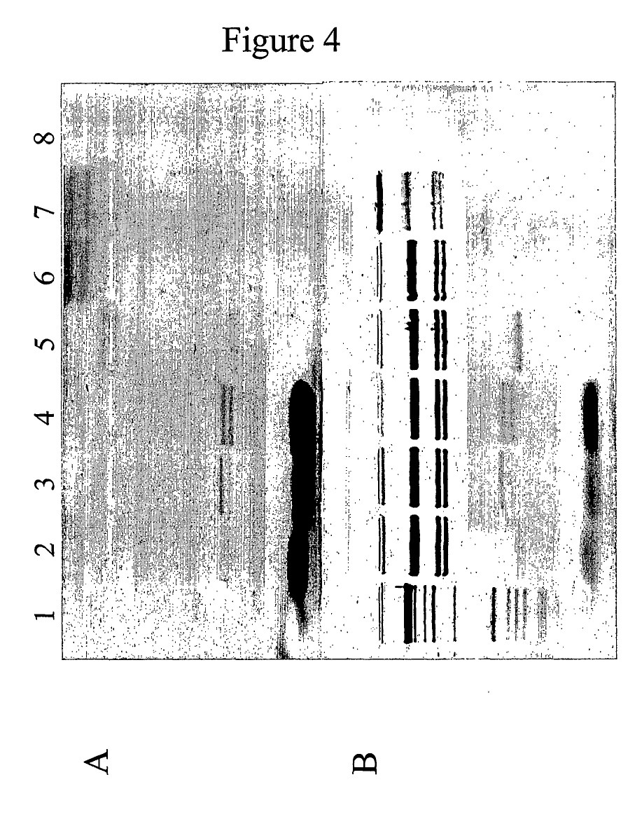

To determine that the oligonucleotides described in this study, both LNA and PNA, had bound to the expected complementary sequence within plasmid DNA, 2.5 ug samples of plasmid DNA from the labelling experiments described in FIG. 2 were subject to restriction enzyme analysis. Briefly, samples were digested with Bsa I and SphI, (8 hours at 37° C. followed by 8 hours at 55° C., respectively under standard conditions, (1×reaction buffer 4, NEB, Beverly, Mass., USA). Samples were then analysed after electrophoresis on 2% agarose/TAE gels without EtBr, as described above. Analysis without EtBr staining is shown in FIG. 4A and after EtBr staining in FIG. 4B. A Bsa I/Sph I digestion of the gWiz plasmid, (6.73 kb, FIG. 1A), produces the following linear DNA fragments of size: 3.66 kb, 1.49 kb, 1.25 kb and 300 bp, (data not shown, FIG. 4B), where the 300 bp Bsa I/Sph I fragment contains all the PNA and LNA oligonucleotide binding sites, (multiple AG motifs), within the bGHpyA region of plasmid gWiz, (FIG. 1A, 50).

Note that all three LNAs, (LNA 2, 4 & 5), shown specific binding to the expected 300 bp fragment as is seen for the GTS PNA control, (FIG. 4 ).

FIG. 4 shows 2% agarose gel electrophoresis, in the absence of ethidium bromide, (EtBr), for analysis of supercoiled plasmid DNAs incubated with rhodamine-labelled LNA or PNA oligonucleotides after overnight digestion at 37° C. with the restriction enzymes Bsa I and Sph I, (New England Biolabs Inc., Beverly, Mass., USA).
(A) Gel analysis on Multi Analyst package on Gel Doc 1000 system, (Bio-Rad Laboratories, California, USA) before EtBr staining,
(B) As (A) after EtBr staining.
1) 1 ug of 1 kb DNA ladder, (Life Technologies Ltd, Paisley, UK)
2) 2.5 ug gWiz plasmid labelled with rhodamine LNA2 at 37° C.
3) 2.5 ug gWiz plasmid labelled with rhodamine LNA4 at 37° C.
4) 2.5 ug gWiz plasmid labelled with rhodamine LNA5 at 37° C.
5) 2.5 ug gWiz plasmid labelled with GTS PNA rhodamine labelling kit
6) 2.5 ug gWiz plasmid labelled with rhodamine OsPNA2 at 37° C.
7) 2.5 ug gWiz plasmid labelled with rhodamine OsPNA2 at 95° C./room temperature.
8) Empty.

The different LNA oligonucleotides being bound to the 300 bp fragment appear to have caused different degrees of retardation of the electrophoretic mobility of this fragment in a 2% agarose gel, (FIG. 4). It is interesting to speculate that this may be related to the numbers of LNA oligonucleotides bound to the 300 b fragment or the degree of occupancy of the multiple binding sites, ie. the most retarded species representing those with the most bound LNA oligonucleotides. The retardation of the 300 bp fragment brought about by binding of the OsPNA 2 oligonucleotide is expected as this PNA is positively charged, also containing glycine and lysine amino acid residues. The faint approximate 3.66 kb band showing rhodamine fluorescence in some lanes, (FIG. 4A ), is most likely to be an approximate 3.96 kb SphI fragment of gWiz, produced by partial restriction enzyme digestion, (ie. Bsa I not fully cutting, FIG. 1A), and would therefore be expected to contain all of the expected oligonucleotide binding sites present in the 300 bp Bsa I/Sph I fragment.

It seems likely that as with the binding of PNA oligonucleotides to supercoiled, double stranded plasmid DNA, binding with LNA oligonucleotides rely upon the expected complementary base pairing rules and do not show detectable non-sequence specific binding.

### EXAMPLE 2

### Behaviour of LNA and PNA Oligonucleotides Bound to Supercoiled Plasmid DNA During the Cartridge Preparation Procedure for PMID or ‘Gene Gun’

Previous attempts to use the commercially available rhodamine-PNA clamp plasmid labelling system., (GTS Inc., San Diego, Calif., USA), in combination with plasmid DNA prepared for PMID with the ‘gene gun’, (Accell XR1, Powderject, Wisconsin, Mass., USA) were not successful. The ‘PNA clamp’ was removed completely during the plasmid/‘gold slurry’ coating procedure, (I. Catchpole, unpublished, see below).

The large scale LNA and PNA oligonucleotide labelled plasmids described above, (FIG. 3), were ethanol precipitated, (51), and resuspended in TE PH 8.0, (10 mM Tris HCl pH 8.0), at 1 ug/ul and were subject to 70-100 ug scale ‘gold slurry’ coating procedures, (see below). Unlabelled pGL3CMV plasmid and pGL3CMV labelled with rhodamine by a chemical method, (direct chemical attachment of rhodamine conjugates to most of the G residues in the plasmid DNA, Mirus Label IT kit, Panvera, Madison, Wis., USA) were also subject to the cartridge preparation procedure, as controls.

Preparation of Plasmid-coated ‘Gold Slurry’ for ‘Gene Gun’ DNA Cartridges

Plasmid DNA, (approximately 1 ug/ul), eg. 100 ug, and 2 μm gold particles, eg. 50 mg, (PowderJect), were suspended in 0.05M spermidine, eg. 100 ul, (Sigma). The DNA was precipitated on to the gold particles by addition of 1M CaCl₂, eg. 100 ul (American Pharmaceutical Partners, Inc., USA). The DNA/gold complex was incubated for 10 minutes at room temperature, washed 3 times in absolute ethanol, eg. 3×1 ml, (previously dried on molecular sieve 3A (BDH)). Samples were resuspended in absolute ethanol containing 0.05 mg/ml of polyvinylpyrrolidone (PVP, Sigma), and split into three equal aliquots in 1.5 ml microfuge tubes, (Eppendorf). The aliquots were for analysis of (a) ‘gold slurry’, (b) eluate-plasmid eluted from (a) and (c) for preparation of gold/plasmid coated Tefzel cartridges for the ‘gene gun’, (see Example 3 below). For preparation of samples (a) and (b), the tubes containing plasmid DNA/‘gold slurry’ in ethanol/PVP were spun for 2 minutes at top speed in an Eppendorf 5418 microfuge, the supernatant was removed and the ‘gold slurry’ dried for 10 minutes at room temperature. Sample (a) was resuspended to 0.5-1.0 ug/ul of plasmid DNA in TE pH 8.0, assuming approx. 50% coating. For elution, sample (b) was resuspended to 0.5-1.0 ug/ul of plasmid DNA in TE pH 8.0 and incubated at 37° C. for 30 minutes, shaking vigorously, and then spun for 2 minutes at top speed in an Eppendorf 5418 microfuge and the supernatant, eluate, was removed and stored at −20° C. The exact DNA concentration eluted was determined by spectrophotometric quantitation using a Genequant II (Pharmacia Biotech).

Analyses of LNA and PNA Conjugated Oligonucleotides/Plasmid Complexes after ‘Gold Slurry’ Preparation

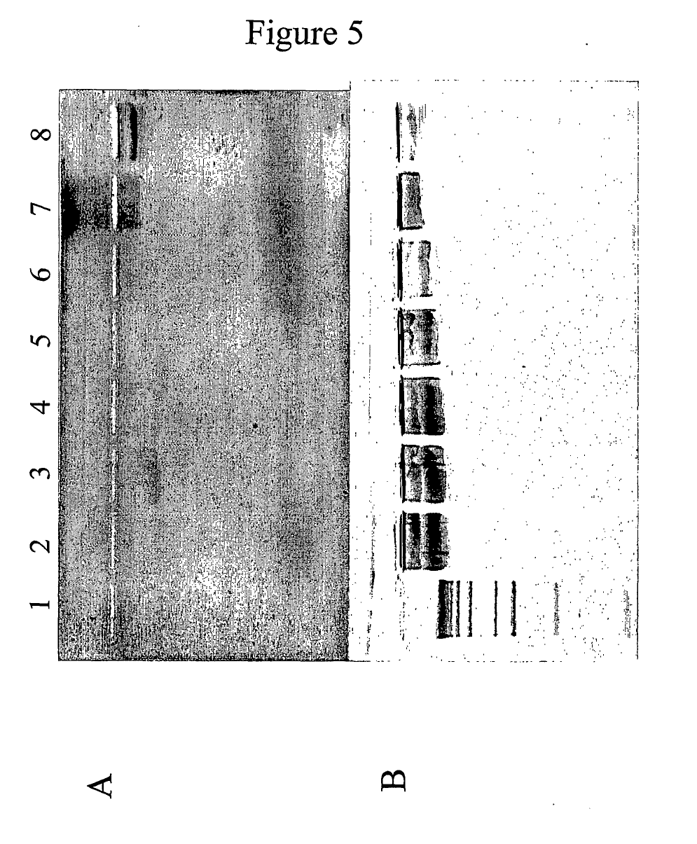

Plasmid samples eluted from ‘gold slurry’, 2.5 ug, were then analysed after electrophoresis on 2% agarose/TAE gels without EtBr, as described, (FIG. 5).

FIG. 5 shows 2% agarose gel electrophoresis, in the absence of ethidium bromide, (EtBr), for analysis of supercoiled plasmid DNAs incubated with rhodamine-labelled LNA or PNA oligonucleotides, subjected to the ‘gold slurry’ preparation procedure, (for PMID cartridge preparation for delivery with the ‘gene gun’), and then eluted from the ‘gold slurry’ with TE PH8.0.
(A) Gel analysis on Multi Analyst package on Gel Doc 1000 system, (Bio-Rad Laboratories, California, USA) before EtBr staining,
(B) As (A) after EtBr staining.
1) 1 ug of 1 kb DNA ladder, (Life Technologies Ltd, Paisley, UK)
2) 2.5 ug gWiz plasmid labelled with rhodamine LNA2 at 37° C.
3) 2.5 ug gWiz plasmid labelled with rhodamine LNA4 at 37° C.
4) 2.5 ug gWiz plasmid labelled with rhodamine LNA5 at 37° C.
5) 2.5 ug gWiz plasmid labelled with GTS PNA rhodamine labelling kit
6) 2.5 ug gWiz plasmid labelled with rhodamine OsPNA2 at 37° C.—post-gold slurry preparation.
7) 2.5 ug gWiz plasmid labelled with rhodamine OsPNA2 at 37° C.—pre-gold slurry preparation
8) 0.8 ug pGL3CMV plasmid labelled with Mirus Label IT rhodamine labelling kit.

A comparative analysis of FIGS. 5A and 5B was used to determine which of the rhodamine-labelled oligonucleotides had remained attached to plasmid DNA after the ‘gold slurry’ preparation. The attachment of LNA 4 appears to be unaffected by the procedure, whereas LNAs 2 and 5 still show a very reduced attachment, to plasmid DNA, as judged by rhodamine fluorescence, (FIG. 5). However, no rhodamine labelled PNA can be seen attached to plasmid DNA in either sample post-‘gold slurry’ preparation, so as seen previously attached PNA oligonucleotides are removed by this procedure. Similar results were seen upon analysis of ‘gold slurry’preparations directly, (data not shown ), so this result is not dependent on the elution procedure.

LNA oligonucleotides appear to be superior to PNA oligonucleotides in withstanding the conditions required for PMID/‘gene gun’ mediated delivery when bound to plasmid DNA. It is likely that one or a combination of the excipients, (ie. spermidine, CaCl₂ and PVP), used to condense plasmid DNA and to coat it on to gold particles interferes with the PNA:DNA hybridisation and removes even sophisticated PNA oligonucleotides such as PNA clamps or ‘bis’ PNA. It seems that LNA:DNA hybridization properties are therefore more robust than PNA:DNA and as long as a sufficient number of LNA:DNA residues are hydrogen bonded, for the application of stability in PMID preparation somewhere in the region of 13 LNA/DNA pairs seems to be minimally required.

### EXAMPLE 3

### Effect on Gene Expression Derived from Supercoiled Plasmid DNA with Bound LNA and PNA Oligonucleotides

If LNA oligonucleotides are to be used to attach functional moieties to plasmid DNA, it would be preferred that LNA binding, per se, did not interfere with gene expression derived from the plasmid. This property has been verified for PNA clamps, and the oligonucleotide binding sites present on plasmid gWIz are in a region of the plasmid where their presence should not obstruct the progression of RNA polymerase enzymes and their co-factors, (50). Although data already described in this work suggested than LNA oligonucleotides bind only to the same cognate PNA-binding sites in gWiz, the affect of this on plasmid delivery and gene expression had not been determined.

Comparative Luciferase Activity of Plasmids with Bound LNA and PNA Oligonucleotides after ‘Gene Gun’ Delivery to MC57 Cells. Cell Culture.

HeLa, human cervical carcinoma cell line, (a gift from C. Kitson, GSK), and MC57, a murine fibrosarcoma cell line, (a gift from P. Gilboy, GSK, 56), were both grown in Dulbecco's modified Eagle's media, (DMEM, Life Technologies), supplemented with 10% fetal calf serum, (FCS), 100 units/ml penicillin, 100 ug/ml streptomycin and 2 mM glutamine, (Life Technologies) at 37° C./5% CO₂.

Preparation of Plasmid-coated ‘Gold Slurry’ for ‘Gene Gun’ DNA Cartridges

Aliquots of plasmid DNA coated ‘gold slurry’ stored under ethanol/PVP, (Example 2), were re-suspended and transferred gently to a scintillation vial containing a sufficient volume of ethanol PVP for efficient Tefzel tube coating, also allowing for a DNA loading rate (DLR) of 2 μg of DNA/mg of gold. Tefzel tubing, (Powderject), which had previously been dried with N₂, was placed inside a tube turner, (Powderject), and the DNA-coated gold was applied to the inner surface of the tubing by centrifugal force. The tubing was cut into 12.5 mm lengths, using a Tefzel tube cutter, (BioRad), and stored with desiccant at 4° C. Typically a preparation of approx. 17 mg of coated ‘gold slurry’ yielded 20-22 cassettes.

In Vitro Transfection by PMID

PMID to MC57 cells was performed using the plate method, (57). The cells were transfected by PMID using an Accell XR1 gene gun, (PowderJect). A helium gas pressure of 250 pounds/inch² (psi) was used to discharge the DNA-coated gold from the cartridge into the cells. 20±5μl of a concentrated cell suspension was evenly spread in the centre of a 6 well plate and transfected while holding the nozzle of the gene gun directly over the cells. Once transfected, 1 ml of medium was added to the cells and they were incubated for 24 hours at 37° C. and 5% CO₂. Cells were washed once with PBS and lysed by addition of 1 ml of passive lysis buffer, (Promega). Six individual 6 well samples were pooled and 40 μl of the pooled lysate (in duplicate) was assayed together with 200 μl of luciferase assay reagent (Promega) in a black 96 well plate, (Nunc). Luciferase activity (RLU) was measured as counts per second in the TopCountNXT HTS scintillation and luminescence counter, (Packard). Total protein concentration was calculated by Coomassie Plus protein assay reagent kit (Pierce) using the manufacturer's protocol. Briefly, 5 μl of cell lysate was assayed together with 145 μl of water (Sigma) and 150 μl of coomassie blue reagent in 96 well flat-bottomed plates (Costar). The absorbance was measured at 595 nm on a Molecular Devices Spectra Max 340. Results were expressed as mean of triplicate samples (μg/ml). Luciferase activity was expressed as relative light units (RLU)/mg of total protein.

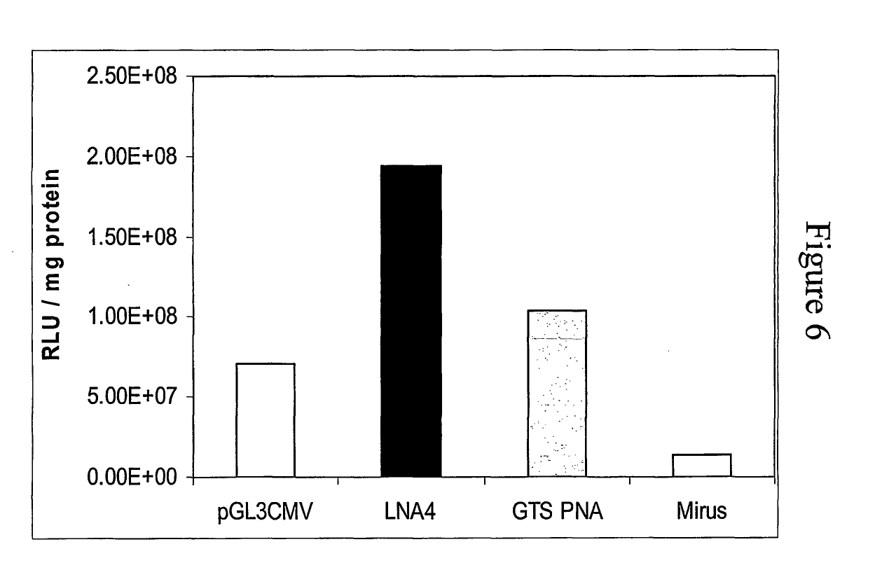

FIG. 6 shows the comparative luciferase expression data from PMID of LNA and PNA labelled luciferase expression plasmids in MC57 cells, 24 hours post-transfection.

FIG. 6 shows comparative luciferase activity, at 24 hours post transfection, of plasmids transiently transfected into MC5-7 cells by gene gun. Note that the Mirus sample is Mirus labelled pGL3CMV plasmid and the samples labelled LNA4 and GTS PNA are LNA and PNA labelled gWiz plasmid respectively. Values represent a mean of six pooled, independent ‘gene gun’ transfections.

This suggests that gWiz plasmid with bound LNA 4 oligonucleotide is at least as transcriptionally active as similar plasmid DNA which had been labelled with GTS PNA, although since this had been removed in the ‘gold slurry’ preparation procedure, (Example 2), the latter sample is representative of unlabelled gWiz plasmid. This can be contrasted with the expression data demonstrated by unlabelled pGL3CMV plasmid compared to Mirus labelled pGL3CMV plasmid, where the latter, chemically modified plasmid displays dramatically reduced gene expression.

In Vitro Transfection by Lipofection with DMRIE-C.

Similar results to those shown for in vitro PMID in MC57 cells were obtained by lipofection into HeLa cells using DMRIE-C, (Life Technologies).

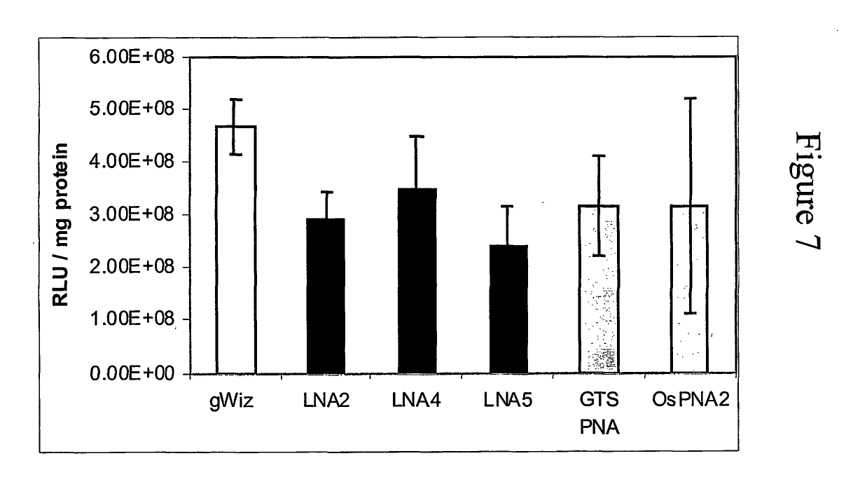

Briefly, plasmids from both the small and the large scale LNA and PNA oligonucleotide labellings, (Example 1, FIGS. 2 and 3, respectively), were transfected, in duplicate into semi-confluent HeLa cells in single wells of a 6 well plate. Mixtures of 0.8 ug of plasmid DNA: 0.8 ul of DMRIE-C were prepared per well, according to manufacturers instructions and these were overlaid onto HeLa cells in 1 ml of Optimem and left overnight, (Life Technologies). Cells were harvested 24 hours post transfection and assayed for luciferase and total protein as described for MC57 above. Comparative luciferase expression data is shown in FIG. 7.

FIG. 7 shows comparative luciferase activity, at 24 hours post transfection of plasmids transiently transfected into HeLa cells using lipofection with DMRIE-C, (Life Technologies). All LNA and PNA labelled samples are based on gWiz plasmid. Values are the mean of four independent transfections, with standard deviations shown.

Similarly, gWiz plasmid with LNA or PNA nucleotides bound showed no significant difference in gene expression compared to unbound gWiz plasmid DNA.

Similar transfections were undertaken into 8 well glass slides, (Nunc), and cells were fixed 24 hours post-transfection with 4% paraformaldehyde. Slides were viewed under the fluorescent microscope, (Diaphot 300 inverted fluorescence microscope with rhodamine filter, Nikon Corporation, Tokyo, Japan), and rhodamine labelled LNA and PNA oligonucleotides, attached to plasmid, could be clearly seen in both nuclear and cytoplasmic compartments for samples derived from all the LNA and PNA oligonucleotides used in this study, (data not shown, 50).

### EXAMPLE 4

### Behaviour of NLS Peptide:PNA Oligonucleotide Conjugates when Bound to Supercoiled Plasmid DNA and Transfected into Mammalian Cells

Plasmids and PNA Oligonucleotides.

The plasmids and PNA oligonucleotides used in this study are all described, (Example 1).

Peptides and Alexa Fluor 568 Labelling.

The peptides used in this study are all listed in Table 2.

TABLE 2

| | Amino | |
|---|---|---|
| Name | acids | Sequence |
| SV40nls | 13 | MPPKKKRKVGSGC |
| AdE | 25 | MAKRARLSTSFNPVYPYEDEKKSSC |
| M9 | 42 | GNQSSNFGPMKGGNFGGRSSGPYGGGGQYFAKPNQ |
| | | GGYGGC |
| FGF | 17 | AAVALLPAVLLALLAPC |

Sv40nls, (7), AdF, (8) and M9 peptides, (9) have been described and were either synthesized in house, (Sv40nls and AdF), on an ABI 433 peptide synthesiser using the 0.25 mmol Fastmoc MonPrvPk method or, (M9), by Research Genetics/Invitrogen BV, (now Invitrogen Life Technologies, Paisley, UK). Peptides were labeled using an Alexa Fluor 568 protein labelling kit, (Molecular Probes, Leiden, Netherlands). Usually 500 ul of peptide at 2 mg/ml in 100 mM sodium phosphate pH 7.2, was labeled with Alexa Fluor 564 dye.
Conjugation of Peptides with Maleimide-PNA Oligonucleotides and Binding to Plasmid DNA.

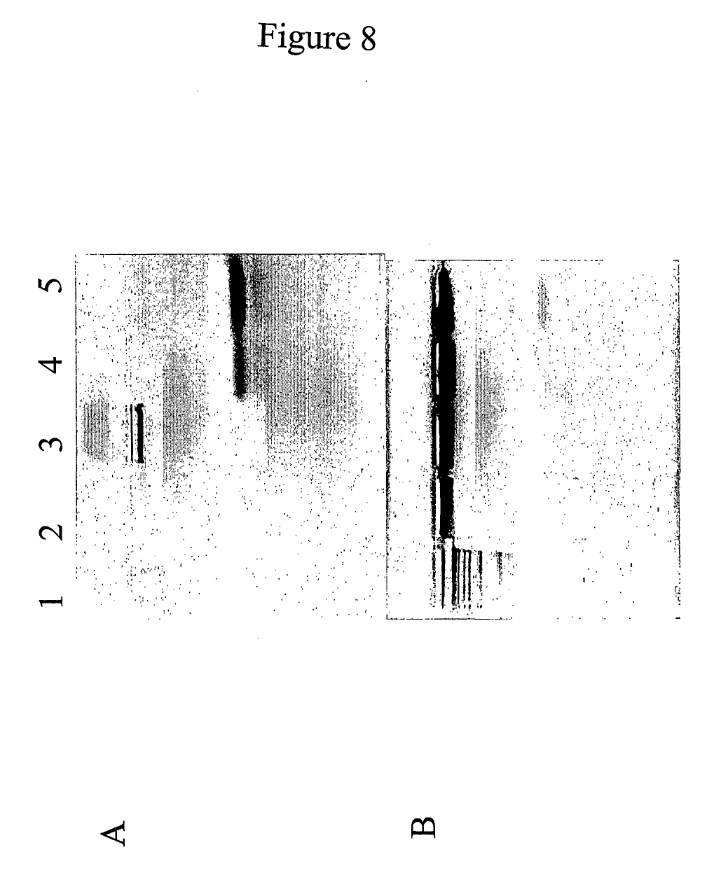

The method for the formation of peptide conjugates with maleimide-PNA, (mal-PNA, GTS Inc., San Diego, USA), was to treat 1 mg of Alexa Fluor 568-labeled peptide with 14 ul of 0.5M TCEP, (Tris (2-carboxyethyl phosphine hydrochloride, Pierce now Perbio, Rockford, Ill., USA), in 100 mM HEPES, (N-[2-Hydroxyethyl]piperazine-N′-[2-ethanesulfonic acid], Sigma Aldrich, Company, Poole, UK.), pH7.0 to 7.2, for 2 hours shaking at room temperature, to generate free sulphydryl groups and activate the maleimide. For mal-PNA either the entire 50 ul sample was similarly TCEP treated or the entire mal-PNA stock was first bound to 134 ug of gWiz, (FIG. 1A), plasmid DNA in 10 mM sodium phosphate buffer, 1 mM EDTA, pH 5.8 at 37° C. overnight and then the plasmid bound mal-PNA was treated with TCEP, as described above. Peptides and mal-PNA, (unbound or plasmid bound), were then mixed whilst shaking at 4° C. overnight for coupling to occur, at a twenty-fold molar excess of peptide. Samples were then further purified by shaking with Sulfolink Coupling gel, (Perbio, Rockford, Ill., USA), for 1 hour at room temperature in a Gene Clean Spin filter tube, (Bio101, Carlsbad, Calif., USA) to remove free uncoupled peptide by free sulphydryl groups, and samples were recovered by spinning for 2 mins at 300 rpm in a microfuge. For peptide coupled mal-PNA samples that were not already bound to plasmid DNA, binding to plasmid gWiz was then undertaken, as described above. Finally, all plasmid samples with bound peptide-malPNA conjugate were separated away from free unbound mal PNA and peptides by microspin S400HR columns, (Amersham Pharmacia Biotech, Little Chalfont, UK), gel exclusion chromatography. The amount of plasmid recovered was then determined by analysis on the Gene Quant RNA DNA Calculator, (Amersham Pharmacia Biotech, Little Chalfont, UK). Samples of 2.5 ug of plasmid DNA were then subject to agarose gel electrophoresis, an example is shown in FIG. 8, where peptides were coupled to mal-PNA subsequent to its binding to plasmid DNA, but similar data was obtained where peptides were conjugated to mal-PNA, prior to binding to plasmid. The data shows that although fluorescent material corresponding to peptide coupled PNA is recovered for agarose gel electrophoresis, only in the case of SV40nls does this remain bound to plasmid DNA, (FIG. 8A).

FIG. 8 shows 2% agarose gel electrophoresis, in the absence of ethidium bromide, (EtBr), for analysis of supercoiled plasmid DNAs incubated with Alexa Fluor 568 labeled peptides conjugated to maleimide GTS PNA oligonucleotides, (Table 1, GTS), and then separated from free unbound PNA oligonucleotide by gel exclusion chromatography through Microspin S400HR columns, (Amersham Pharmacia Biotech, Little Chalfont, UK). Peptide sequences are listed in Table 2.
(A) Gel analysis on Multi Analyst package on Gel Doc 1000 system, (Bio-Rad Laboratories, California, USA), before EtBr staining,
(B) As (A) after EtBr staining.
1) 1 ug of 1 kb DNA ladder, (Life Technologies Ltd, Paisley, UK)
2) 2.5 ug gWiz plasmid incubated with maleimide GTS PNA
3) 2.5 ug gWiz plasmid incubated with SV40nls peptide/maleimide GTS PNA
4) 2.5 ug gWiz plasmid incubated with AdF peptide/maleimide GTS PNA
5) 2.5 ug gWiz plasmid incubated with M9 peptide/maleimide GTS PNA

Similar experiments performed with Alexa Fluor 568 SV40nls peptide alone, (data not shown), show that even in the absence of PNA this peptide can bind to plasmid DNA. It can therefore be concluded that although AdF and M9 peptide coupled mal-PNA can remain associated to plasmid DNA sufficiently well to pass through a S400HR column, the interaction is unstable and peptide-mal-PNA conjugate and plasmid separate upon agarose gel electrophoresis.

HeLa Cell Transfection by Electroporation.

HeLa cells were grown as described in Example 3. A HeLa cell transfection assay based upon electroporation of a large number of HeLa cells, approximately 2×10⁷ per sample, with limiting amounts of a plasmid DNA expressing the luciferase gene, was devised such that the level of luciferase expression produced is linearly proportional to plasmid DNA dose, (data not shown). For this system a linear relationship between gWiz plasmid DNA dose and luciferase expression was demonstrated for 1 to 10 ug of plasmid DNA over a time period of at least 48 hours. Following such a protocol, 1 ug amounts of gWiz plasmid bound to the three NLS peptide-mal PNA conjugates described above, ie. either containing SV40nls, AdF or M9 peptide were electroporated into HeLa cells. Briefly, HeLa cells were harvested and half-of the contents of a confluent 1×175cm² flask of cells were spun down at 1400 rpm in a Sorvall RT6000D bench top centrifuge, (DuPont, UK), for 6 mins, washed with Optimem, (Invitrogen Life Technologies, Paisley, UK), and then finally resuspended in 500 ul of Optimem in 0.4 cm Gene Pulser electroporation cuvette, (BioRad, Hemel Hempstead, UK), cells and plasmid DNA were then mixed. Each cuvette was then subjected to a single pulse of 300V, 960 uF on a Gene Pulser II electroporator. Cells were then left for 5 minutes to recover and were then resuspended in 10 mls of full media, 5 mls was used to seed a 1×25 cm² flask, (for harvesting at 5 hours post-transfection), and the remaining cells were seeded into a 1×75 cm² flask, (for harvesting at 21 hours post-transfection). Cells were washed once with PBS and lysed by addition of 1 ml of passive lysis buffer, (Promega), 40 μl of the pooled lysate, (in duplicate), was assayed together with 200 μl of luciferase assay reagent (Promega) in a black 96 well plate, (Nunc). Luciferase activity (RLU) was measured as counts per second in the TopCountNXT HTS scintillation and luminescence counter, (Packard). Total protein concentration was calculated by Coomassie Plus protein assay reagent kit, (Perbio), using the manufacturer's protocol. Briefly, 5 μl of cell lysate was assayed together with 145 μl of water (Sigma) and 150 μl of coomassie blue reagent in 96 well flat-bottomed plates (Costar). The absorbance was measured at 595 nm on a Molecular Devices Spectra Max 340. Results were expressed as mean of triplicate samples (μg/ml). Luciferase activity was expressed as relative light units (RLU)/mg of total protein.

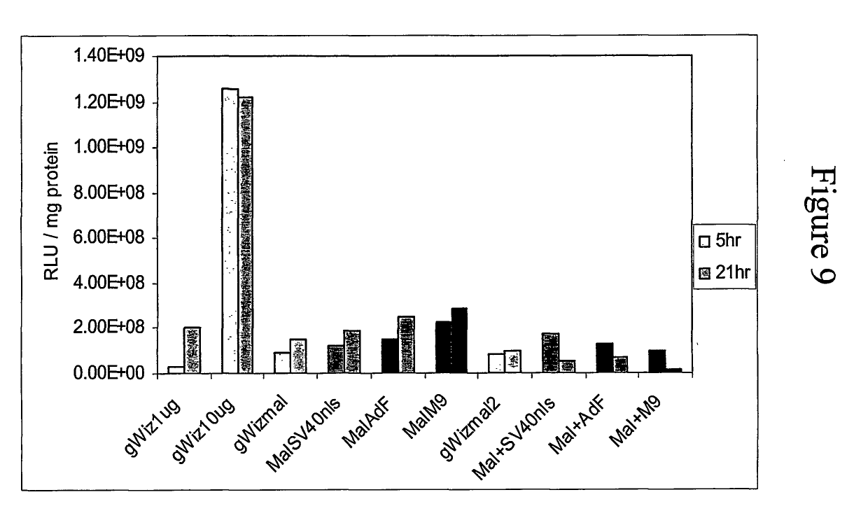

The data from such an experiment is shown in FIG. 9.

FIG. 9 shows comparative luciferase activity, at 5 & 21 hours post transfection, of plasmids transiently transfected into HeLa cells by electroporation. All PNA labeled samples are based on gWiz plasmid; gWizmal and gWizmal2=1 ug gWiz plasmid labeled with maleimide coupled PNA, (GTS); MalSV40nls, MalAdF and MalM9=1 ug of gWiz plasmid labeled with maleimide coupled PNA to which one of the peptides: SV40nls, AdF or M9, respectively, had been attached prior to plasmid binding; Mal+SV40nls, Mal+AdF and Mal+M9=1 ug of gWiz plasmid labeled with maleimide coupled PNA to which one of the peptides: SV40nls, AdF or M9, respectively, were subsequently attached after plasmid binding.

Here, the attachment of NLS peptides as conjugates via mal-PNA, (GTS), to gWiz plasmid DNA show no significant increases in luciferase expression compared to gWiz plasmid DNA alone or gWiz plasmid bound to mal-PNA. If the peptide-malPNA conjugates are pre-formed prior to binding of plasmid DNA there is no obvious inhibition of gWiz derived luciferase expression, whereas if either of all three peptides tested are conjugated to pre-plasmid-bound mal-PNA, gWiz derived luciferase expression is inhibited. Similar data was obtained in similar experiments using mal-PNA again or SPDP, (N-Succinimidyl 3-[2-pyridyldthio]propionate)-PNA (GTS, California, USA). Taken together with the data in FIG. 8, the reduced stability of binding of NLS peptide-PNA oligonucleotide conjugates can lead to reductions and not enhancements of gene expression. A more stable plasmid binding system than PNA oligonucleotides is required for analyzing the effects of conjugated NLS peptides on plasmid derived gene expression.

### EXAMPLE 5

### Investigation of LNA Oligonucleotide Sequence Requirements and Conditions for Binding to Supercoiled Plasmid DNA Plasmids

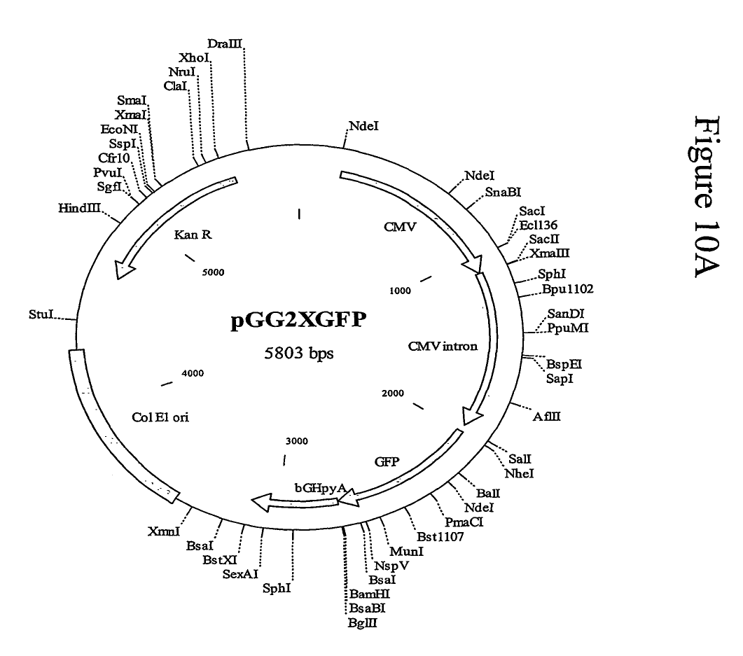

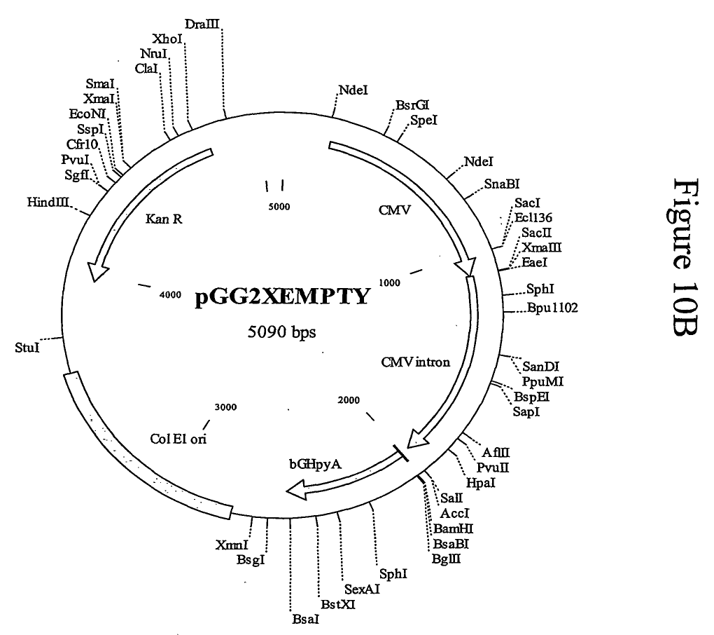

The plasmids used in this study have been described in FIGS. 1 and 10. FIG. 10A shows plasmid pGG2XGFP a GFP expression vector, (Gene Therapy Systems [GTS], Inc., San Diego, Calif., USA), this contains multiple AG motifs within the DNA sequence of the bGHpyA region of the plasmid and multiple AAGG motifs within the DNA sequence 5′ to the CMV promoter, (GTS Catalogue 2002, 50). Plasmid pGG2XEMPTY is an expression vector, derived from pGG2XGFP by deletion of the GFP gene, but retaining a polylinker for the insertion of a gene of interest, to be expressed under the control of the CMV promoter. To construct plasmid pGG2XEMPTY from plasmid pGG2XGFP, the latter was digested with the restriction enzymes Nhe I and BamH I, (New England Biolabs, [NEB], Hitchin, Herts., UK), to delete the region encoding the GFP gene and the remaining 5.1 kb plasmid fragment was gel purified, treated with Klenow DNA Polymerase, (NEB), and ligated together using T4 DNA ligase, (NEB), prior introduction in to E. coli, (51). Bacterial cells containing plasmid pGG2XEMPTY were identified by standard procedures, (51), and large scale plasmid DNA preparations were as described in Example 1.

LNA and PNA Oligonucleotides used in this Study

Some of the LNA oligonucleotides used in this study have been described before, (Table 1, Example 1). Novel LNA and PNA oligonucleotides are listed in Table 3.

TABLE 3

lists oligonucleotide
sequences used in this study.

| | No. | | |
|---|---|---|---|
| | of | | |
| Name | sites | Description | Sequence |
| 5876 | 6 | 13mer 100% | 5′-NH₂-CTCTCTCTCTCTC-3′ |
| | | LNA | |
| 5877 | 5 | 14mer 100% | 5′-NH₂-CCTTCCTTCCTTCC-3′ |
| | | LNA | |
| 5827 | 6 | 13mer 100% | 5′-NH₂-GAGAGAGAGAGAG-3′ |
| | | LNA | |
| 5875 | 6 | 11mer 100% | 5′-NH₂-CTCTCTCTCTC-3′ |
| | | LNA | |
| 5747 | 8 | 9mer ‘bis’ | 5′-NH₂-CtCtCtCtC-XXX-Ct |
| | | 50% LNA | CtCtCtC-3′ |
| 6563 | 6 | 11mer 100% | 5′-TAMRA-CTCTCTCTCTC-3′ |
| | | LNA | |
| 11701 | 5 | 14mer 100% | 5′-NH₂-GGAAGGAAGGAAGG-3′ |
| | | LNA | |
| PTOCpG | 6 | 21mer | 5′ [symbol] tG |
| | | DNA13mer | AGAGAGAGAGAG-3′ |
| | | LNA | |
| PTOGpC | 6 | 21mer | 5′ [symbol] tG |
| | | DNA13mer | AGAGAGAGAGAG-3′ |
| | | LNA | |
| 5′SHGA | 6 | 13mer 100% | 5′-S-S-GAGAGAGAGAGAG-3′ |
| | | LNA | |
| PNA223 | 6 | 13mer 100% | 5′-ROX-O-O-gCTCTCTCTCTC |
| | | PNA | TCk |
| PNA234 | 6 | 13mer ‘bis’ | 5′-ROX-O-O-gCTCTCTCTCTC |
| | | 100% PNA | TC-OOO-CTCTCTCTCTCTCk |
| RevGG2 | 1 | 22mer 100% | 5′Cy5/ggaaggaagttaggaagg |
| B | | DNA | aagg- 3′ |
| kh2 | 4 | 19mer 100% | 5′Fl- gagagagagagagagag |
| | | DNA | ag- 3′ |
| kh3 | 3 | 18mer 100% | 5′Fl- ggaaggaaggaaggaag |
| | | DNA | g- 3′ |
| CPG1826 | — | 20mer 100% | 5′- [symbol] |
| | | PTO | |
| CPG1745 | — | 20mer 100% | 5′- [symbol] |
| | | PTO | |

LNA residues are displayed in bold upper case, DNA residues are shown in bold lower case with PTO residues additionally italicised, PNA and amino acid residues are shown in italics, (ordinary text, non-bold, with PNA bases in upper case). Number of sites refers to the maximum number of theoretical oligonucleotide binding sites present on either the gWiz or pGG2XGFP, (FIG. 10a) plasmid. X=‘PEG spacer’-9-O-Dimethoxytrityl-triethylene glycol, 1-[(2-cyanoethyl)-(,N-diisopropyl)]-phosphoramidite, spacer phosphoramidate 9, (Glen Research, USA); O=8-amino-3,6-dioxaoctanoic acid linker, g=glycine, l=lysine, Fl=Fluorescein, NH₂=5′-amino-modifier C12 phosphoramidite spacers, (Glen Research, USA), PTO=phosphorothioate, S-S=Thiol modifier, C6 S-S phosphoramidate, (Glen Research, USA).

All LNA oligonucleotides were synthesized by Proligo LLC, Colorado, USA. The majority were made with 5′-amino-modifier C12 phosphoramidite spacers, (Glen Research, USA), to allow for labelling with Alexa Fluor dyes, (Molecular Probes, Netherlands), or heterobifunctional linkers, eg. Maleimide or SPDP, (Perbio, USA). Most are 100% LNA monomers, but LNA 5747, (Table 3), is 50% LNA and 50% DNA. This ‘bis’ LNA oligonucleotide was made as an analogue of the ‘bis’ PNA clamps described in Example 1, (50, 52), and could only be efficiently synthesized as a 50:50 mix of LNA and DNA residues. The PNAs described in Table 3 were all purchased from Oswel DNA Service, Southampton, UK.

Alexa FluorDye Labelling of LNA Oligonucleotides.

LNA oligonucleotides were 5′ end labeled at the primary amine group NH₂ with Alexa Fluor dyes: 568, 647 or 350 (Molecular Probes, Leiden, Netherlands). Usually 25 to 100 ug of LNA oligonucleotide was labeled. To ensure efficient labelling, the oligonucleotide was extracted three times with an equal volume of chloroform and then ethanol precipitated and resuspended in a very small volume of Millipore purified water, (1 to 4 ul). Labelling reactions were based upon 0.1M sodium tetraborate buffer, pH8.5, using Alexa Fluor dyes made up in dimethyl suphoxide, (DMSO, Sigma Aldrich Company, UK) and were incubated overnight at room temperature, with shaking. Post-labelling, Alexa Fluor labeled LNA oligonucleotides were purified away from free dye by ethanol precipitation and resuspended in Millipore purified water at 20 to 200 pmoles/ul, concentration, verified by Gene Quant, (Amersham Pharmacia Biotech, Little Chalfont, UK).

Analysis of Conditions for Binding of LNA and PNA Oligonucleotides to Supercoiled Plasmid DNA

Plasmid labelling with LNA oligonucleotides was usually performed under the conditions described in Example 1: 10 mM phosphate buffer, 1 mM EDTA, pH 7.0 for 16 hours at 37° C., with a twenty-fold molar excess of oligonucleotide over the maximum number of PNA/LNA binding sites in the plasmid. Initially using the LNA oligonucleotides LNA2, LNA4 and LNA5, Table 1, and binding to plasmid gWiz, FIG. 1A, a range of conditions were evaluated including: reducing pH from 7.0 to 5.8, the addition of sodium chloride to 100 mM and the presence of 3M TMAC, (Tetramethyl ammonium chloride, Sigma Aldrich, UK). Incubation time at 37° C. was also reduced to 3 hours. For all the conditions described above all three LNAs could be detected as bound to plasmid by the assay described in Example 1, (data not shown). From this analysis, the most efficient binding appeared to be LNA4, (100% LNA 13mer), in 10 mM phosphate buffer, 1 mM EDTA, pH 5.8 for 16 hours at 37° C.

In a similar study the PNA oligonucleotides described in Table 3 were also evaluated: PNA223 was found not to bind supercoiled gWiz plasmid DNA under similar conditions to that used for the analysis of binding of the LNA oligonucleotides. However PNA234 was found to bind plasmid but only in 10 mM phosphate buffer, 1 mM EDTA, pH 5.8 after incubation for 16 hours at 37° C., (data not shown).

Further analysis of LNA oligonucleotide plasmid binding conditions were undertaken with LNA oligonucleotides: LNA2, LNA4 and LNA5, Table 1. For this analysis, incubations of LNA oligonucleotides were performed at ‘low’, (5 pmoles of oligo./ug of plasmid DNA), and ‘high’, (5 pmoles of oligo./ug of plasmid DNA), doses. The temperature and time of annealing was also evaluated in this analysis where incubation times of 1 hour at room temperature or 37° C. and 16 hours at 4° C. were evaluated. For all of these analyses the buffer conditions were 10 mM phosphate buffer, 1 mM EDTA, pH 5.8. For all the conditions described, all three LNAs could be detected as bound to plasmid by the assay described in Example 1, (data not shown).

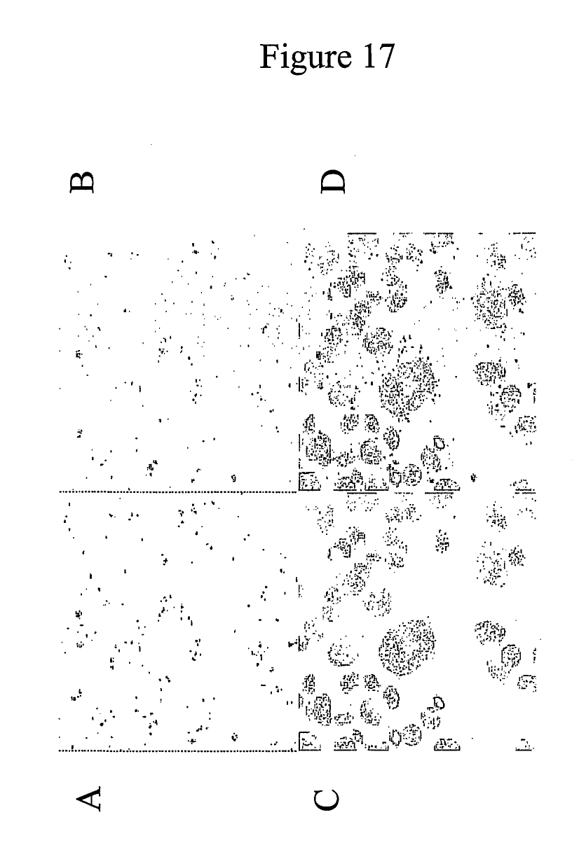

In addition LNA oligonucleotide binding to plasmid DNA has also been detected in 100 mM sodium phosphate pH7.0 and also in the presence of 1.25 mM cobalt chloride and 100 mM potassium cacodylate, (data not shown). More strikingly LNA oligonucleotides have been shown to still bind supercoiled plasmid DNA that has been chemically labeled with the Mirus Label IT nucleic acid labelling kit, (Panvera, Madison, USA) or containing bound intercalating TOTO-1 iodide dye, (Molecular Probes, Leiden, Netherlands), see Example 8 and FIG. 17 for details.

Analysis of Novel LNA Oligonucleotides for Binding to Supercoiled Plasmid DNA

In order to test whether or not the plasmid binding properties of LNA oligonucleotides were dependent upon specific base sequences such as (CT)ₙ, Table 1, or the properties are more broadly applicable to different base sequences a number of different LNA oligonucleotides were synthesized, Table 3. These were all based around DNA sequences at the repeat binding sites for PNA found in the Gene Grip series of plasmids, (GTS, San Diego, Calif., USA). Site 1 being found in gWiz and pGGGFP, (FIGS. 1A and 1B), and based upon a (CT)ₙ repeat motif, (complementary strand [GA]ₙ), and site 2, based upon (CCTT)ₙ, (complementary strand [GGAA]ₙ), being found in pGG2XGFP and pGG2XEMPTY, (FIGS. 10A and 10B), both the latter two plasmids also contain site 1, (GTS catalogue 2002).

FIG. 10 shows plasmids used in this study
(A) pGG2XGFP is a GFP expression vector, (Gene Therapy Systems [GTS], Inc., San Diego, Calif., USA), this contains multiple AG motifs within the DNA sequence of the bGHpyA region of the plasmid and multiple AAGG motifs within the DNA sequence 5′ to the CMV promoter, (GTS Catalogue 2002, 50).
(B) pGG2XEMPTY is an expression vector, derived from pGG2XGFP by deletion of the GFP gene, but retaining a polylinker for the insertion of a gene of interest, to be expressed under the control of the CMV promoter.

LNA 5876, a (CT)ₙ based 100% 13mer LNA, Table 3, is equivalent to the rhodamine labeled version, LNA4, Table 1. Given that the 100% LNA 13mer, but not the 100% LNA 9mer was stable during the ‘gold slurry’ preparation for ‘gene gun’delivery, Example 2, it was decided to synthesise an intermediate 11mer (CT)ₙ based LNA, either 5875 or the rhodamine labeled 6563, Table 3. Oligonucleotides based upon 13-14mer 100% LNAs were made for the complementary strand to site 1, ie. 5877, Table 3, and also to each DNA strand of site 2, ie. 5877 and 11701, respectively. Also a ‘bis’ LNA, 5747, as described above, was made based upon the (CT)ₙ motif, to test the idea that a ‘bis’ based LNA clamp might be more stable than simple oligonucleotides, allowing both Watson-Crick and Hoogsteen base pairing, in an analogous manner to the PNA clamps described earlier.

All of the rhodamine or Alexa Fluor-568 labeled LNA oligonucleotides described above and in Table 3, showed binding to supercoiled plasmids containing their cognate binding sites, (data not shown).

Verification of Novel LNA Oligonucleotide Binding to Cognate Binding Sites within Plasmid DNA.

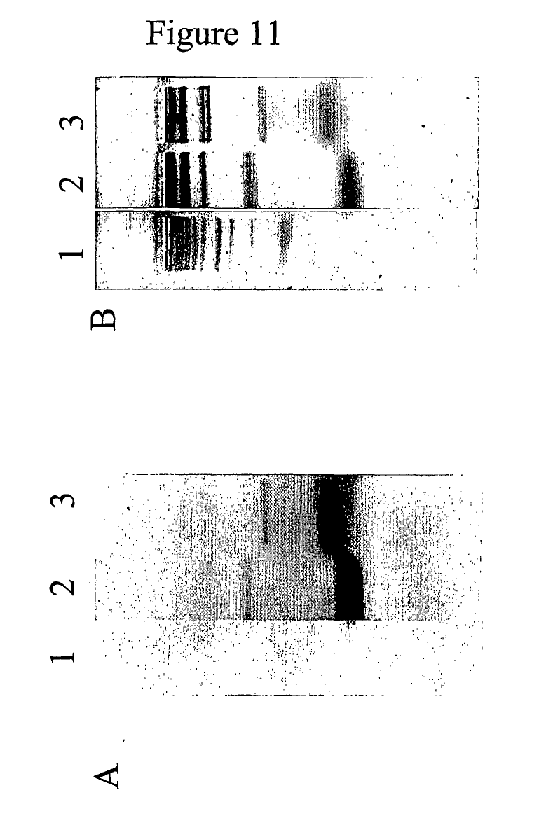

To determine that the LNA oligonucleotides described above had bound to the expected complementary sequence within plasmid DNA, 2.5 ug samples of plasmid DNA from the labelling experiments described above, were subject to restriction enzyme analysis. For rhodamine or Alexa Fluor 568 labeled LNAs: 5876, 5827, 5875, 5747 and 6563 binding was to plasmid gWiz overnight at 37° C. Samples were digested with Bsa I and SphI, (8 hours at 37° C. followed by 8 hours at 55° C., respectively under standard conditions, (1× reaction buffer 4, NEB, Beverly, Mass., USA). Samples were then analysed after electrophoresis on 2% agarose/TAE gels without EtBr, as described, Example 1. For Alexa Fluor 568 labeled LNAs: 5877 and 11701, binding was to plasmids pGG2XGFP or pGG2XEMPTY overnight at 37° C. Samples were digested with Nde I, (16 hours at 37° C.), under standard conditions, (1× reaction buffer 4, NEB, Beverly, Mass., USA). Samples were then analysed after electrophoresis on 2% agarose/TAE gels without EtBr, as described, Example 1. An example of such analysis is shown in FIG. 11, where agarose gel electrophoresis without EtBr staining is shown in FIG. 11A and after EtBr staining in FIG. 11B.

FIG. 11 shows 2% agarose gel electrophoresis, in the absence of ethidium bromide, (EtBr), for analysis of supercoiled plasmid DNAs incubated with Alexa Fluor 568-labeled LNA oligonucleotides after overnight digestion at 37° C. with the restriction enzymes Bsa I and Sph I, or NdeI (New England Biolabs Inc., Beverly, Mass., USA).
(A) Gel analysis on Multi Analyst package on Gel Doc 1000 system, (Bio-Rad Laboratories, California, USA) before EtBr staining,
(B) As (A) after EtBr staining.
1) 1 ug of 1 kb DNA ladder, (Life Technologies Ltd, Paisley, UK)
2) 2.5 ug pGG2XGFP plasmid labeled with Alexa Fluor 5877 at 37° C., cut Nde I
3) 2.5 ug gWiz plasmid labeled with Alexa Fluor 5827 at 37° C., cut Bsa I and Sph I

A Bsa I/Sph I digestion of the gWiz plasmid, (6.73 kb, FIG. 1A), produces the following linear DNA fragments of size: 3.66 kb, 1.49 kb, 1.25 kb and 300 bp, (data not shown, FIG. 11B), where the 300 bp Bsa I/Sph I fragment contains all the LNA oligonucleotide binding sites, (multiple CTₙ or GAₙ motifs), within the bGHpyA region of plasmid gWiz, (FIG. 1A, 50). Note that all five rhodamine or Alexa Fluor labeled LNAs: 5876, 5827, 5875, 5747 and 6563 showed specific binding to the expected 300 bp fragment, the example of 5827 is shown in FIG. 11. A Nde I digestion of the pGG2XGFP plasmid, (5.80 kb, FIG. 10A), produces the following linear DNA fragments of size: 3.76 kb, 1.56 kb, and 491 bp, (data not shown, FIG. 11B), where the 491 bp Nde I fragment contains all the LNA oligonucleotide binding sites, (multiple CCTTₙ or GGAANₙ motifs), within the region just 5′ to the CMV promoter of plasmid pGG2XGFP, (FIG. 10A, GTS Catalogue 2002). ). Note that both Alexa Fluor labeled LNAs: 5877 and 11701, showed specific binding to the expected 491 bp fragment, the example of 5877 is shown in FIG. 11.

It should also be noted that plasmid binding was not seen if LNA oligonucleotides were similarly incubated with supercoiled plasmids that did not contain the cognate binding site, eg. LNA 5877 binds to plasmid pGG2XGFP, (FIG. 10A), but not to pGGGFP, (FIG. 1B), (data not shown).

### EXAMPLE 6

### Behaviour of a Range of LNA Oligonucleotide Sequences and PNA Oligonucleotides Bound to Supercoiled Plasmid DNA During the Cartridge Preparation Procedure for PMID or ‘Gene Gun’

LNA and PNA Oligonucleotides.

The LNA and PNA oligonucleotides used in this study are listed in Table 1 and Table 3 and were either synthesized with rhodamine attached at the 5′ end or were 5′ Alexa Fluor 568 labeled, as described, Example 5. Specifically the LNA and PNA oligonucleotides compared in this analysis were: rhodamine labeled: 6563, LNA4 and PNA234 and Alexa Fluor 568 labeled: 5747, 5827 and 5877.

Preparation of Plasmid-coated ‘Gold Slurry’ for ‘Gene Gun’ DNA Cartridges

Basically the method described in Example 2 was followed with some minor modifications. Large scale annealings of labeled LNA or PNA oligonucleotides to 25 ug of plasmids gWiz or pGG2XGFP were performed overnight at 37° C. A 2.5 ug sample was removed as a ‘pre-gold slurry preparation’ control and the remaining 22.5 ug of plasmid DNA was coated on to 2 μm gold particles, ie. 11.25 mg, (PowderJect), suspended in 0.05M spermidine, eg. 100 ul, (Sigma). The DNA was precipitated on to the gold particles by addition of 1M CaCl₂, eg. 100 ul (American Pharmaceutical Partners, Inc., USA) and the procedure followed as described in Example 2. It was attempted to elute all 22.5 ug of the plasmid from each sample in 20 ul, as described, but as this was unsuccessful, the gold slurry preparations containing the remainder of the 22.5 ug of gWiz plasmid were also analysed by agarose gel electrophoresis as described below.

Analysis of LNA and PNA Conjugated Oligonucleotides/Plasmid Complexes After ‘Gold Slurry’ Preparation

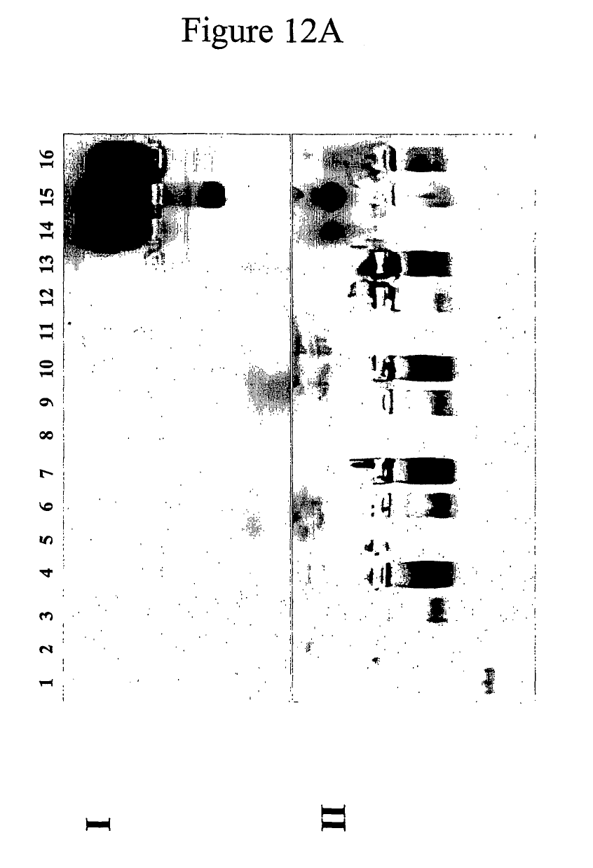

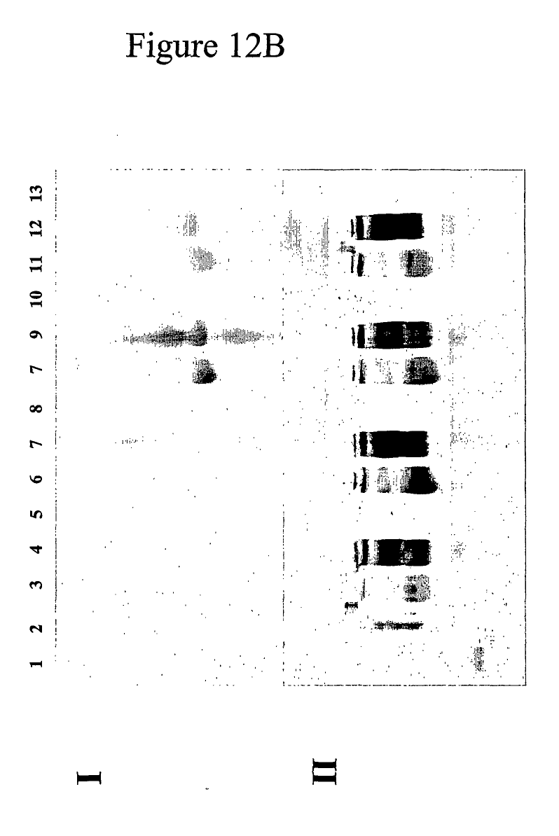

Plasmid samples that were attempted to be eluted from ‘gold slurry’, direct ‘gold slurry’ samples and 2.5 ug plasmid samples with bound fluorescently labeled PNA and LNA oligonucleotides, (‘pre-gold slurry preparations’), were then analysed after electrophoresis on 2% agarose/TAE gels without EtBr, as described, (FIGS. 12A and 12B). A comparative analysis of FIG. 12A: I and II and FIG. 12B: I and II was used to determine which of the fluorescently-labeled oligonucleotides had remained attached to plasmid DNA after the ‘gold slurry’ preparation. Since plasmid did not elute from the slurry under these conditions, the results are taken from analysis of ‘gold slurry’ preparations directly. The data obtained was also compared with that described in Example 2 and shown in FIG. 5.

FIG. 12 shows 1.5% agarose gel electrophoresis, in the absence of ethidium bromide, (EtBr), for analysis of supercoiled plasmid DNAs incubated at 37° C. with either high, (approx. 40 pmol/ug plasmid), or low, (5 pmol/ug plasmid), rhodamine (6563, LNA4, PNA234), or Alexa Fluor 568, (5747, 5827, 5877) labeled LNA or PNA oligonucleotides, subjected to the ‘gold slurry’ preparation procedure, (for PMID cartridge preparation for delivery with the ‘gene gun’), and either directly loaded onto the gel or then eluted from the gold slurry with TE pH 8.0. N.B. Under these conditions plasmids did not elute and were retained on the ‘gold slurry’.
(A) Gel analysis for 6563, LNA4 and PNA234
(I) Gel analysis using the Labworks 4.0 package on the UVP EpiChemi Darkroom Bio Imaging System, 302 nm UV-ethidium bromide filter, (UVP, Cambridge, UK), before EtBr staining.
(II) As (I) after EtBr staining.
1) 1 ug of 100 bp DNA ladder, (Life Technologies Ltd, Paisley, UK)
2) Eluate of pGG2XGFP plasmid labeled with LNA4 (40 pmol/ug plasmid)—post-gold slurry preparation
3) pGG2XGFP plasmid labeled with LNA4 (40 pmol/ug plasmid)—pre-gold slurry preparation
4) Gold slurry preparation of pGG2XGFP plasmid labeled with LNA4 (40 pmol/ug plasmid)
5) Eluate of pGG2XGFP plasmid labeled with LNA4 (5 pmol/ug plasmid)—post-gold slurry preparation
6) pGG2XGFP plasmid labeled with LNA4 (5 pmol/ug plasmid)—pre-gold slurry preparation
7) Gold slurry preparation of pGG2XGFP plasmid labeled with LNA4 (5 pmol/ug plasmid)
8) Eluate of pGG2XGFP plasmid labeled with 6563 (40 pmol/ug plasmid)—post-gold slurry preparation
9) pGG2XGFP plasmid labeled with 6563 (40 pmol/ug plasmid)—pre-gold slurry preparation
10) Gold slurry preparation of pGG2XGFP plasmid labeled with 6563 (40 pmol/ug plasmid)
11) Eluate of pGG2XGFP plasmid labeled with 6563 (5 pmol/ug plasmid)—post-gold slurry preparation
12) pGG2XGFP plasmid labeled with 6563 (5 pmol/ug plasmid)—pre-gold slurry preparation
13) Gold slurry preparation of pGG2XGFP plasmid labeled with 6563 (5 pmol/ug plasmid)
14) Eluate of pGG2XGFP plasmid labeled with PNA234 (40 pmol/ug plasmid)—post-gold slurry preparation
15) pGG2XGFP plasmid labeled with PNA234 (40 pmol/ug plasmid)—pre-gold slurry preparation
16) Gold slurry preparation of pGG2XGFP plasmid labeled with PNA234 (40 pmol/ug plasmid)
(B) Gel analysis for PNA234, 5747, 5827 and 5877
(I) Gel analysis using the Labworks 4.0 package on the UVP EpiChemi Darkroom Bio Imaging System, (UVP, Cambridge, UK), before EtBr staining.
(II) As (1) after EtBr staining.
b 1) 1 ug of 100 bp DNA ladder, (Life Technologies Ltd, Paisley, UK)
2) Eluate of pGG2XGFP plasmid labeled with PNA234 (5 pmol/ug plasmid)—post-gold slurry preparation
3) pGG2XGFP plasmid labeled with PNA234 (5 pmol/ug plasmid)—pre-gold slurry preparation
4) Gold slurry preparation of pGG2XGFP plasmid labeled with PNA234 (5 pmol/ug plasmid)
5) Eluate of pGG2XGFP plasmid labeled with 5747 (36 pmol/ug plasmid)—post-gold slurry preparation
6) pGG2XGFP plasmid labeled with 5747 (36 pmol/ug plasmid)—pre-gold slurry preparation
7) Gold slurry preparation of pGG2XGFP plasmid labeled with 5747 (36 pmol/ug plasmid)
8) Eluate of pGG2XGFP plasmid labeled with 5827 (40 pmol/ug plasmid)—post-gold slurry preparation
9) pGG2XGFP plasmid labeled with 5827 (40 pmol/ug plasmid)—pre-gold slurry preparation
10) Gold slurry preparation of pGG2XGFP plasmid labeled with 5827 (40 pmol/ug plasmid)
11) Eluate of pGG2XGFP plasmid labeled with 5877 (15 pmol/ug plasmid) -post-gold slurry preparation
12) pGG2XGFP plasmid labeled with 5877 (15 pmol/ug plasmid)—pre-gold slurry preparation
13) Gold slurry preparation of pGG2XGFP plasmid labeled with 5877 (15 pmol/ug plasmid)

All the LNA oligonucleotides tested in this analysis are retained on the plasmid upon the ‘gold slurry’ preparation, including 6563, the 11mer 100% LNA oligonucleotide. For the application of stability in PMID preparation this reduces the minimal LNA sequence requirements to 11 LNA/DNA base pairings. Of the LNA oligonucleotides, 5827 shows the best binding and seems the least disrupted by the ‘gold slurry’ preparation procedure. The ‘bis’ 9mer 50% LNA, 50% DNA oligonucleotide 5747 is retained by the plasmid in the ‘gold slurry’ preparation procedure, in contrast to the 9mer LNA2, (Table 1, Example 2, FIG. 5). However, this LNA was difficult to synthesise, (Proligo LLC, personal communication), and appears to offer no advantage over the other 13 to 14mer 100% LNA oligonucleotides.

The PNA oligonucleotide, PNA 234, did show a small degree of retention of plasmid binding after the ‘gold slurry’ preparation, in contrast to previous PNA oligo nucleotides, (Table 1, Example 2, FIG. 5). However, even the 13mer ‘bis’ PNA with additional amino acids to improve stability was outperformed by all the simple 100% LNA oligonucleotides tested in this analysis.

This confirms the superiority of LNA over PNA oligonucleotides in withstanding the conditions required for PMID/‘gene gun’ mediated delivery when bound to plasmid DNA.

### EXAMPLE 7

### Mechanism of Binding of LNA Oligonucleotides to Supercoiled Plasmid DNA

Binding of (CT)ₙ-based LNA Oligonucleotides to Plasmid DNA at High and Low Oligonucleotide Concentrations.

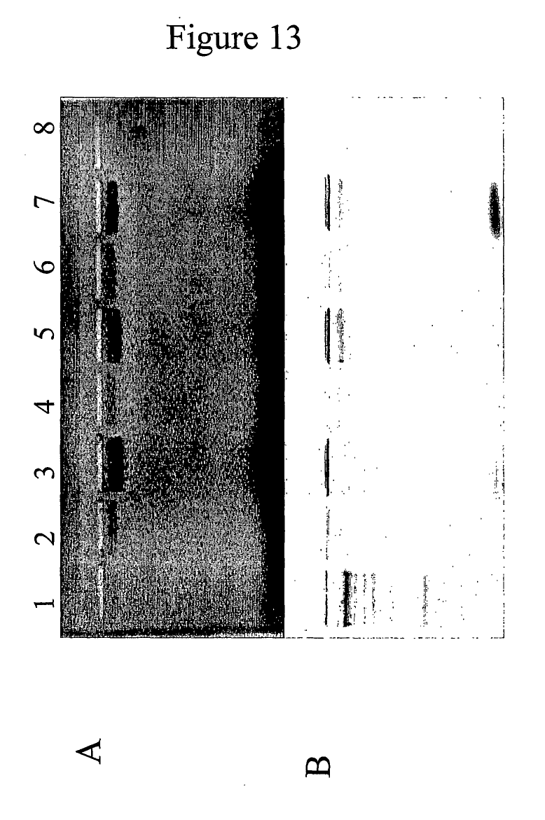

Initially attempts were made to at look differences in the binding of fluorescently labeled LNA oligonucleotides to supercoiled plasmid DNA at low, (5 pmoles/ug DNA), and high, (40 pmoles/ug DNA), oligonucleotide concentrations, as part of the analysis of binding conditions described in Example 5. Analyses of LNAs based upon the (CT)ₙ sequence: LNA2, LNA4 and LNA5 for binding to gWiz plasmid DNA are shown in FIG. 13.

FIG. 13 shows 2% agarose gel electrophoresis, in the absence of ethidium bromide, (EtBr), for analysis of supercoiled plasmid DNAs incubated with rhodamine-labeled LNA oligonucleotides with either high, (approx. 40 pmol/ug plasmid), or low (5 pmol/ug plasmid).
(A) Gel analysis on Multi Analyst package on Gel Doc 1000 system, (Bio-Rad Laboratories, California, USA), before EtBr staining,
(B) As (A) after EtBr staining.
1) 1 ug of 1 kb DNA ladder, (Life Technologies Ltd, Paisley, UK)
2) 2.5 ug gWiz plasmid labeled with rhodamine LNA2 (5 pmol/ug plasmid) at 37° C.
3) 2.5 ug gWiz plasmid labeled with rhodamine LNA2 (40 pmol/ug plasmid) at 37° C.
4) 2.5 ug gWiz plasmid labeled with rhodamine LNA4 (5 pmol/ug plasmid) at 37° C.
5) 2.5 ug gWiz plasmid labeled with rhodamine LNA4 (40 pmol/ug plasmid) at 37° C.
6) 2.5 ug gWiz plasmid labeled with rhodamine LNA5 (5 pmol/ug plasmid) at 37° C.
7) 2.5 ug gWiz plasmid labeled with rhodamine LNA5 (40 pmol/ug plasmid) at 37° C.
8) Empty

Clearly all three LNA oligonucleotides show markedly increased binding to plasmid DNA at the the high compared to the low oligonucleotide concentration, (FIG. 13A). The number of theoretical binding sites for the oligonucleotides range from 4 to 8, for longer to shorter oligonucleotides. However, the markedly increased intensity of plasmid bound LNA oligonucleotides, in each case, at the higher concentration, suggest that two potential mechanisms for LNA oligonucleotide binding may be in operation, perhaps even doubling the numbers of plasmid bound LNAs at high compared to low oligonucleotide concentrations. This could be achieved by both Watson Crick and Hoogsteen base pairing at high LNA oligonucleotide concentrations and lead to triplexes of LNA:DNA:LNA, similar to those described for ‘bis’ PNAs, (50, 52). Note that similar behaviour could not be demonstrated for other LNA oligonucleotides that were not based upon the sequence (CT)ₙ, (data not shown), so this may be a feature of oligonucleotides based upon this repeating polypyrimidine sequence, which are thought to Hoogsteen base pair more readily, (50, 52).

DNA Sequencing Assay to Demonstrate Strand Displacement in Supercoiled Plasmids upon LNA Oligonucleotide Binding.

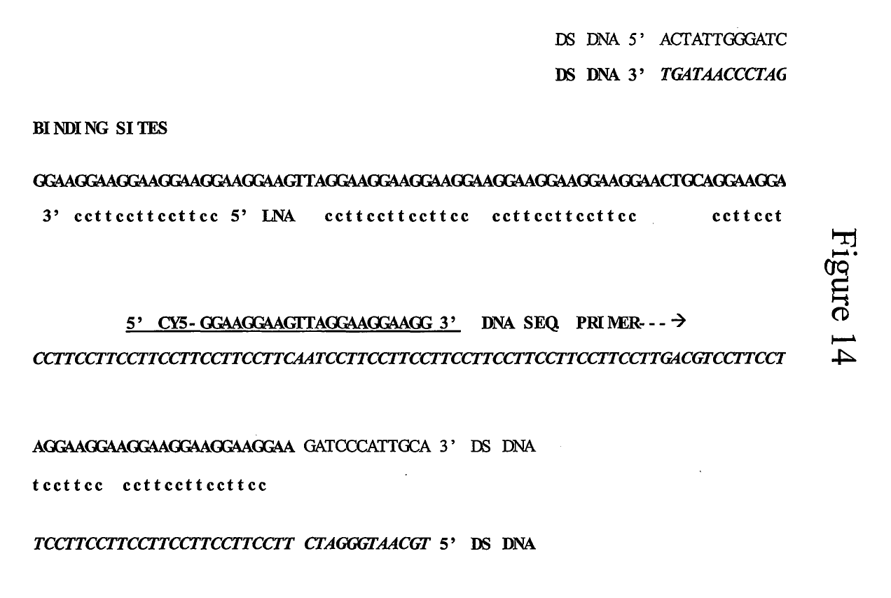

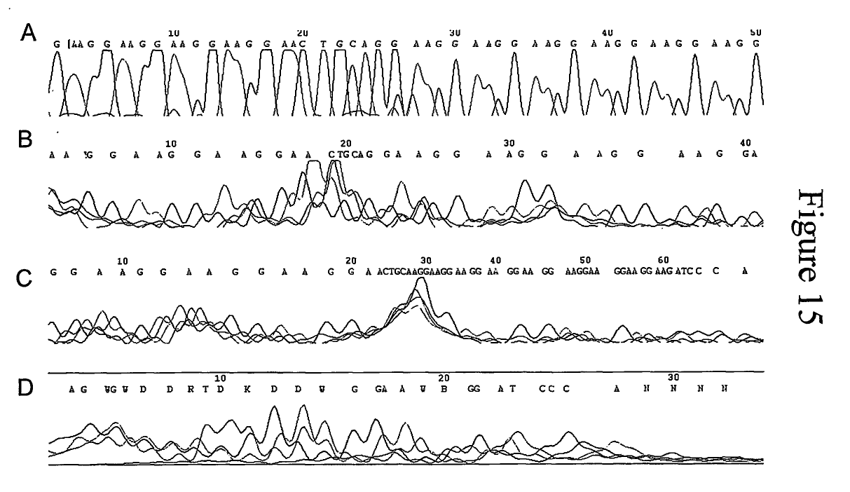

In order to resolve conclusively whether or not LNA oligonucleotides binding to plasmid DNA causes strand displacement of the unbound DNA strand, a single stranded DNA sequencing assay was established. This was based upon a modification of similar methods that have been described to demonstrate PNA oligonucleotide based strand invasion and displacement, (53, 76). The modified procedure was to use double stranded supercoiled plasmid DNA, (in the presence or absence of a bound LNA oligonucleotide), as a template for a DNA sequencing procedure that is optimal only for single stranded DNA. The plasmid chosen for this analysis was pGG2XGFP, (FIG. 10A), and the LNA chosen as a ‘strand displacing agent’ was 5877, (Table 3). An optimal DNA sequencing primer, (RevGG2B, Table 3), was designed, (both with and without a 5′ CyS label), and verified for good quality sequencing across the repeat region at binding site 2 in pGG2XGFP, (see FIG. 14), by standard ‘big dye’PCR-based thermocycle sequencing, (see FIG. 15A).

FIG. 14 shows the DNA sequencing strategy at binding site 2 from plasmid pGG2XGFP, (GGAA)n binding site motif, in the presence of LNA 5877 using the DNA sequencing primer CyS RevGG2B, (Table 3).

A large scale LNA binding was set up, as described previously, so that LNA 5877, (low concentration: 5 pmoles/ug), was bound to 25 ug of plasmid pGG2XGFP and any unbound LNA oligonucleotide was removed by passing through a microspin S400HR column, (Amersham Pharmacia Biotech). Plasmid pGG2XGFP, (with and without bound LNA), was then subject to a modified single stranded DNA sequencing protocol using the AutoRead Sequencing Kit, (Amersham Pharmacia Biotech), with the Cy5 labeled RevGG2B DNA primer and T7 DNA Polymerase. For this analysis, the dose of input template plasmid DNA was varied from 1 ug to 3 ug and the annealing temperature reduced to either 37° C. or 42° C., but the time for annealing was extended to 30 minutes. This was to maximize sequence specific binding of the DNA sequencing primer to any displaced single stranded DNA regions under conditions that should not disrupt the double stranded nature of the plasmid. The sequencing reactions were then run and analysed on a Visible Genetics DNA Sequencer and the data is shown in FIG. 15.

FIG. 15 shows DNA sequence data, chromatograms deciphered manually, for plasmid pGG2XGFP in the presence or absence of LNA 5877 sequenced using a Cy5RevGG2B primer with the ALF single stranded DNA sequencing kit, (Amersham Pharmacia Biotech, Little Chalfont, UK), and visualized on a Visible Genetics DNA Sequencer, (Visible Genetics, Cambridge, UK). A control of plasmid pGG2XGFP subject to double strand DNA sequencing by the ‘big dye’ PCR based thermocycle sequencing protocol and visualized on an ABI 3700 DNA Analyzer, (ABI, Warrington, Cheshire, UK) is also shown, (A).
(A) pGG2XGFP, (no LNA bound, double stranded DNA)—double strand DNA sequenced
(B) pGG2XGFP +LNA 5877—single strand DNA sequenced, 3 ug template at 42° C. annealing temperature
(C) pGG2XGFP +LNA 5877—single strand DNA sequenced, 1 ug template at 37° C. annealing temperature
(D) pGG2XGFP, (no LNA bound, double stranded DNA)—single strand DNA sequenced, 1 ug template at 37° C. annealing temperature

Using the known DNA sequence of the region, (FIG. 14), the DNA sequence obtained in FIGS. 15B and 15C, for plasmid with LNA bound, could be interpreted by eye and is clearly the correct DNA sequence for specific binding of the DNA sequencing primer to its cognate binding site. For plasmid without LNA bound, FIG. 15D, only random, non-specific uninterpretable signal can be seen. This data clearly suggests that, at low LNA oligonucleotide concentrations, (5 pmoles/ug DNA), the LNAs bind to the correct binding sites, probably by strand invasion and Watson Crick base pairing, leading to strand displacement allowing single stranded DNA sequencing of the displaced strand.

Demonstration of LNA Oligonucleotide Driven Strand Invasion of Supercoiled Plasmid DNA by Binding of Fluorescently Labeled DNA Oligonucleotides to the Displaced Strand.

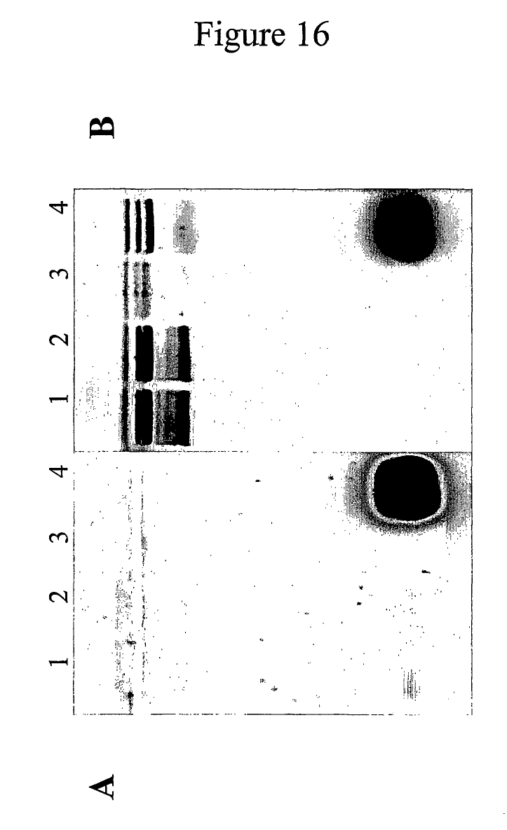

To provide a more simple assay to demonstrate strand displacement of the complementary DNA strand upon LNA oligonucleotide binding to plasmid DNA, a modification of a described method to bind DNA oligonucleotides to the displaced strand was employed, (78). Briefly LNA oligonucleotides were bound to 25 ug of plasmid at low and high concentrations, as described, and any unbound LNA was removed by a microspin S400HR column. Then a fluorescein labeled DNA oligonucleotide of complementary sequence to the LNA and therefore capable of binding the displaced DNA strand was incubated with the plasmid at 37° C. for 45 minutes in standard LNA oligonucleotide binding buffer, (10 mM sodium phosphate, pH7.1, 1 mM EDTA), at a concentration of 40 pmoles/ug DNA, and again free unbound oligonucleotide was removed with a microspin S400HR column. Binding of the fluorescently labeled DNA oligonucleotide to the plasmid DNA strand displaced by LNA binding was analysed by agarose gel electrophoresis. An example is shown in FIG. 16, showing detection of LNA 5867 mediated strand displacement, of plasmid gWiz, with the DNA oligonucletide KH2, (Table 3).

FIG. 16 shows 1.5% agarose gel electrophoresis, in the absence of ethidium bromide, (EtBr), for analysis of supercoiled plasmid DNA incubated with high, (40 pmol/ug plasmid), and low, (5 pmol/ug plasmid) concentrations of LNA overnight at 37° C., (unbound LNA was removed by S400HR spin column gel filtration, Amersham Pharmacia Biotech), and subsequently incubated with a fluorescein labeled DNA primer, (40 pmol/ug plasmid, MWG Biotech AG, Germany).
(A) Gel analysis using the Labworks 4.0 package on the UVP EpiChemi Darkroom Bio Imaging System, 302 nm uv, EtBr filter (UVP, Cambridge, UK), before EtBr staining.
(B) As (A) after EtBr staining.
1) 2.5 ug gWiz plasmid incubated with high concentration of LNA 5876 and KH2
2) 2.5 ug gWiz plasmid incubated with low concentration of LNA 5876 and KH2
3) Empty
4) 2.5 ug gWiz plasmid incubated with KH2 Very similar data was obtained using plasmid pGG2XGFP as template and LNA 5877 binding and strand displacing DNA at binding site 2 so that DNA oligonucleotide KH3, (Table 3), can bind the displaced strand, (data not shown). The latter experiment is analogous to how the DNA sequencing strategy was performed and DNA sequence data was obtained, (FIG. 15) and confirms the validity of both analyses.

Taken together, the DNA sequence data and the DNA oligonucleotide binding data, to the displaced strand, suggest that the major mechanism of binding of LNA oligonucleotides, even at the low oligonucleotide concentration, is by Watson-Crick base pairing via strand invasion and strand displacement.

### EXAMPLE 8

### LNA Oligonucleotides Remain Attached to Supercoiled Plasmid DNA in Mammalian Cells, Post Transfection and Allow Plasmid Derived Gene Expression

Cell Culture and Transfection of CHO Cells

CHO K1 cells were maintained in Iscove's Modified Dulbecco's Medium, supplemented with 10% foetal calf serum, (FCS), 100 units/ml penicillin, 100 ug/ml streptomycin, 2 mM glutamine, MEM non-essential amino acids and HT supplement, (Life Technologies). CHO K1 cells were grown to 80% confluence in 8 well glass chamber slides, (Lab Tech, Nalge Nunc, Int.), washed twice with 400 ul Optimem per well and transfected with I00 ul of plasmid DNA: cationic lipid complex, (200-500 ng plasmid DNA at a DNA: Transfast™,[Promega], ratio of 1 ug to 6 ul), in Optimem. Transfection mix was left in contact with the cells for 24 hours and either cells were washed and fixed, or for longer time points, transfection mix was removed, cells were washed once with phosphate buffered saline, (PBS), and replaced with full growth media for further incubation.

Labelling Plasmid DNA with Mirus Label IT Nucleic Acid Labelling Kits

Plasmid DNA was usually labeled with the Mirus Label IT, (Panvera, Madison, USA), fluorescein labelling kit, though use of the rhodamine labelling kit has been described, Example 3. Plasmid was usually labeled at the 100 ug scale in a total volume of 125 ul at 37° C. for one hour, following manufacturer's instructions, and free dye was removed by standard ethanol precipitation procedures, (51). DNA was resuspended at about 1 ug/ul in water for LNA binding and transfection experiments.

Labelling Plasmid DNA with TOTO-1 Nucleic Acid Dye

Plasmid was usually labeled with Toto-1, (Molecular Probes, Leiden, Netherlands), at the 100 ug scale in 1×TAE, containing Toto-1 dye, (600 ul of 0.5 uM Toto-1 in TAE), in a total volume of 800 ul at room temperature for one hour and free dye was removed by standard ethanol precipitation procedures, (51). DNA was resuspended at about 1 ug/ul in water for LNA binding and transfection experiments.

Cell Fixing and Confocal Fluorescent Microscope Analysis

Cells, grown on 8 well glass chamber slides, (Nalge, Nunc), were washed twice with PBS, then fixed for 10 minutes by incubating with 4% paraformaldehyde and then washed once more with PBS. The plastic chamber and rubber seal were removed, cells were dried and then mounted in Vectorshield mounting medium for fluorescence containing DAPI, to enable visualization of cell nuclei under a UV light source, (Vector Labs., Burlington, Calif., 9401U, USA.) and a No. 1 thickness glass cover slip, (Esco, Erie Scientific, Portsmouth, N.H., USA), was sealed on top using nail varnish, (white ice, No7, Boots PLC, Nottingham, England).

Fixed cells were analysed by confocal fluorescence microscopy using a Leica TCS NT confocal microscope, utilising Argon (UV, 351 and 364 nm), Argon (visible, 476 mn and 488 nm), Krypton (visible, 568 nm) and Helium/Neon (visible, 633 nm) lasers linked by fibre optics to a confocal scanner. The scanner was attached to the side port of a Leitz DM IRBE inverted microscope, controlled by a computer and samples were viewed using a 63× water immersion lens. Images were analysed by the TCS image analysis package.

Demonstration that Plasmid-bound Single and Multiple LNA Oligonucleotides Co-localise with Plasmid DNA in CHO Cells Post-tranfection.

In order to demonstrate that LNA oligonucleotides when bound to plasmid DNA remain associated with the plasmid after transfection into mammalian cells, transfection experiments were performed upon CHO cells with plasmid pGG2XEMPTY, (FIG. 10B), which had been previously labeled either with a Mirus fluorescein labeling kit, (Panvera), or with bound Toto-1, (Molecular Probes), dye. In an initial experiment, Mirus labelled plasmid, (25 ug), was bound with rhodamine labelled 6563 LNA, (overnight at 37° C., 10 mM sodium phosphate, 1 mM EDTA pH 8.0, 40 pmoles oligo./ug, Table 3), and after separation through an S400HR spin column to remove unbound LNA, the labelled plasmid with bound LNA was transfected into CHO cells. An example of confocal analysis of such CHO cells, fixed 36 hours post-transfection is shown in FIG. 17. This clearly demonstrates that there is a 100% correlation or co-localisation of the plasmid derived fluorescein signal and the 6563 LNA derived rhodamine signal. This suggests that the bound LNA is retained on the plasmid after lipid based transfection into CHO cells.

FIG. 17 shows co-localisation in CHO cells, at 36 hours post-transfection, of LNA bound to plasmid, visualised under a confocal microscope, (Leica TCS NT).
(A) PGG2xEMPTY plasmid labeled with Minis Label IT fluorescein labelling kit visualised on a FITC channel (Argon laser, 488 mn excitation)
(B) LNA 6563, (labeled with rhodamine), visualised on a TRITC channel (Krypton laser, 568 nm excitation)
(C) DAPI stained nuclei, visualised under UV light (Argon laser, 351+364 nm excitation)
(D) Overlay of the three different channels: (A)+(B)+(C)

In a very similar experiment it was demonstrated that 3 LNA oligonucleotides of different sequences binding to two different sites within plasmid pGG2XEMPTY also show co-localisation with plasmid DNA after transfection into CHO cells, (data not shown). Plasmid pGG2XEMPTY, (25 ug, FIG. 10B), which had been previously labeled either with a Mirus fluorescein labeling kit, (Panvera), or with bound Toto-1, (Molecular Probes), dye was bound with LNA4, (labeled with rhodamine, Table 1), overnight at 37° C., 10 mM sodium phosphate, 1 mM EDTA pH 8.0, (40 pmoles oligo./ug), free unbound LNA was removed after separation through an S400HR spin column. Subsequently, LNA 5877, labeled with Alexa Fluor 647 and LNA 5827, labeled with Alexa Fluor 350, were bound in turn with any free oligonucleotide being removed at each stage by an S400HR spin column separation. Plasmid DNA was then transfected into CHO cells which were fixed 48 hours post transfection for analysis under the confocal microscope. For the four channel confocal microscope analysis:
(i) PGG2xEMPTY plasmid labeled with Toto-1, (Molecular Probes, Leiden, Netherlands), was visualised on a FITC channel, (Argon laser, 488 nm excitation),
(ii) LNA4, (labeled with rhodamine),was visualised on a TRITC channel, (Krypton laser, 568 nm excitation),
(iii) LNA 5877 labeled with Alexa Fluor 647 was visualised on the Far red/Cy5 channel, (Helium-Neon laser, 633 nm excitation),
(iv) LNA 5827 labeled with Alexa Fluor 350 was visualised under UV light, (Argon laser, 351+364 nm excitation). Colocalisation of all four colours could be clearly demonstrated in CHO cells, (data not shown), suggesting that all three LNA oligonucleotides remain bound to plasmid DNA post-transfection. Note that LNA 4, (Table 1), and 5827, (Table 2), bind to similar sites within plasmid pGG2XEMPTY, but on complementary DNA strands, whereas 5877, (Table 2), binds to an unrelated DNA sequence. Note also that it had been previously shown by restriction mapping data similar to that shown in FIGS. 4 & 11, (for LNA 4 and 5877 respectively), that these two LNA oligonucleotides could be simultaneously and specifically bound to plasmid DNA at their cognate binding sites, (data not shown).
   Demonstration that GFP Expression Plasmids with Bound Fluorescent LNA Oligonucleotides Co-localise to CHO Cells Post-tranfection and Show GFP Expression

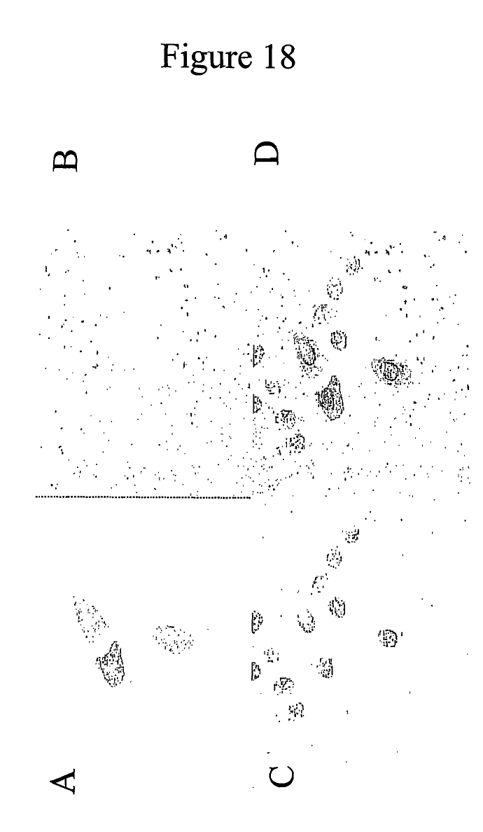

Confocal microscopy experiments were performed in order to demonstrate that when LNA oligonucleotides are bound at their cognate binding sites within the plasmids described in this work, (eg. pGG2XGFP, FIG. 10A, GTS Catalogue, 2002, GTS, California, USA), plasmids that contain bound LNA can demonstrate expression of a plasmid encoded gene. Plasmid pGG2XGFP, (25 ug), was bound with rhodamine labelled 6563 LNA, (overnight at 37° C., 100 mM sodium phosphate, 1 mM EDTA pH 8.0, 40 pmoles oligo./ug, Table 3), and after separation through an S400HR spin column to remove unbound LNA, the labelled plasmid with bound LNA was transfected into CHO cells grown upon an 8 well glass slide. Cells were fixed and processed, as described above, 66 hours post transfection and expression of green fluorescent protein, (GFP) and detection of plasmid bound rhodamine-labelled LNA were detected by confocal microscopy, see FIG. 18.

FIG. 18 shows co-localisation in CHO cells, at 66 hours post-transfection of LNA bound to plasmid, expressing Green Fluorescent Protein, (GFP), visualised under a confocal microscope (Leica TCS NT).
(A) PGG2XGFP plasmid expressing GFP visualised on a FITC channel (Argon laser, 488 nm excitation)
(B) LNA 6563, (labeled with rhodamine), visualised on a TRITC channel (Krypton laser, 568 mu excitation).
(C) DAPI stained nuclei, under UV light (Argon laser, 351+364 nm excitation)
(D) Overlay of the three different channels: (A)+(B)+(C)

CHO cells expressing GFP derived from transfected pGG2XGFP plasmid that also contained a rhodamine signal from bound 6563 LNA were readily detectable. These results provide additional validation at the molecular level to add to the experimental data described in Example 3, demonstrating that gene expression can occur when LNA oligonucleotides are bound to the plasmids described in this work.

### EXAMPLE 9

### Attachment of CpG Based Immune Adjuvants to Supercoiled Plasmid DNA with LNA Oligonucleotides and Assay for Immune Adjuvant Effect in Vitro in RAW264.7 Cells

LNA and PTO, (Phosphorothioate), Oligonucleotides, Plasmid DNA and Endotoxin Testing.

The LNA oligonucleotides PTOCpG and PTOGpC are described in Table 3 and were synthesized by Proligo LLC, Colorado, USA. Briefly both oligonucleotides were synthesized to contain twenty phosphorothioate residues at the 5′ end, followed by a single DNA phosphoramidate residue to facilitate enzymatic cleavage and release of free CpG after administration, followed at the 3′ end by thirteen LNA residues. The LNA residues were as described for LNA 5827, (Table 3), but without the modified amino group. The phosphorothioate component of these oligonucleotides are based upon the described CpG adjuvant oligonucleotide 1826, (80, 81), for PTOCpG and its control 1745 where the CpG motifs have been mutated for GpC, (81). The phosphorothioate oligonucleotides CPG1826, (80, 81) and CPG1745, (81, Table 3), are simply the 100% phosphorothioate component of oligonucleotides PTOCpG and PTOGpC, respectively, and were synthesized by MWG-Biotech AG, Ebersberg, Germany.

Plasmid DNA, gWiz, (FIG. 1A), and pGG2XGFP, (FIG. 10B), was prepared by the Qiagen Endofree Plasmid Maxi Kit according to manufacturer's instructions, (Qiagen, GmbH, Germany).

The absence of endotoxin was confirmed in all oligonucleotides and plasmids used in this study by measuring the endotoxin levels using either the Biowhittaker QCL-1000 LAL kit, (Biowhittaker Inc., Walkersville, USA), or the Pyrochrome LAL kit, (associates of Cape Cod Inc., Falmouth, Mass., USA). Assays were performed according to manufacturer's instructions. Endotoxin levels for all plasmids and oligonucleotides used in this study were less than 0.1 EU, (endotoxin units),/ug DNA.

Chemical Labelling of Oligonucleotides with the Ulysis Kit

Oligonucleotides were labeled using the Ulysis nucleic acid labelling kit, (Molecular Probes, Leiden, Netherlands), containing the Alexa Fluor 488 fluorescent dye. Briefly, oligonucleotides were generally labeled at the 5 ug scale in 105 ul total volume at 80° C. for 15 minutes and the reaction was stopped by plunging on ice and free dye was removed by standard ethanol precipitation procedures, (51). Oligonucleotides were then resuspended in water at around 20-25 pmoles/ul for plasmid binding experiments.

Binding of ‘Hybrid’ PTO/LNA Oligonucleotides to Plasmid DNA

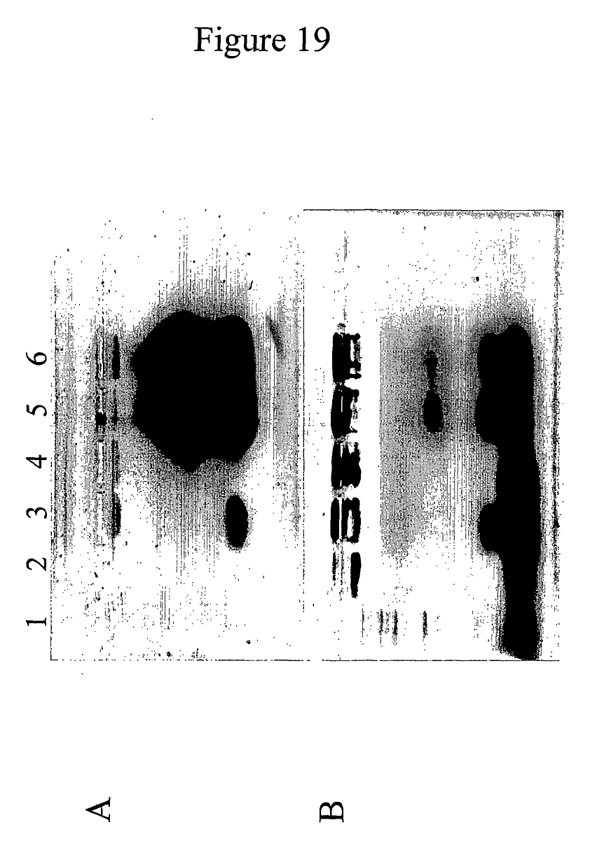

Plasmid pGG2XEMPTY was bound with either PTOCpG or PTOGpC, (overnight at 37° C., 10 mM sodium phosphate, 1 mM EDTA pH 8.0, Table 3). Briefly, 2.5 ug of plasmid DNA was bound with approximately 90 pmoles of Ulysis Alexa Fluor 488 labeled oligo./ug of DNA, and the resulting products were analysed on an agarose gel, see FIG. 19.

FIG. 19 shows 2% agarose gel electrophoresis, in the absence of ethidium bromide, (EtBr), for analysis of supercoiled plasmid DNAs incubated with LNA oligonucleotides labeled with either Alexa Fluor 568 at a 5′ NH₂ group or chemically labeled at the N⁷ G residue with a Ulysis Alexa Fluor 488 labelling kit, (Table 3, Molecular Probes, Leiden, Netherlands).
(A) Gel analysis using the Labworks 4.0 package on the UVP EpiChemi Darkroom Bio Imaging System, 302 nm uv, SYBR gold filter (UVP, Cambridge, UK), before EtBr staining.
(B) As (A) after EtBr staining.
1) 1 ug of 1 kb DNA ladder, (Promega, Southampton, UK)
2) 2.5 ug pGG2xEMPTY plasmid incubated with 5′ Alexa Fluor 568 labeled 5827 at 37° C.
3) 2.5 ug pGG2xEMPTY plasmid incubated with Ulysis Alexa Fluor 488 labeled 5827 at 37° C.
4) 2.5 ug pGG2xEMPTY plasmid incubated with Ulysis Alexa Fluor 488 labeled PTOCpG at 37° C.
5) 2.5 ug pGG2xEMPTY plasmid incubated with Ulysis Alexa Fluor 488 labeled PTOGpC at 37° C.

In both cases fluorescently labeled oligo could be detected as being bound to plasmid DNA in a similar manner to fluorescently labeled control LNAs such as 5827 that are based upon the LNA component of both PTOCpG and PTOGpC, (FIG. 19). This clearly demonstrates that the 5′ extension of a 13mer 100% LNA oligonucleotide by 21 phosphorothioate and DNA residues does not interfere with the LNA's ability to bind to plasmid DNA. Additionally the Ulysis labeling kit is known to add fluorescent dye molecules to the N7 atom of the purine ring of preferentially guanine, but also adenine residues. Since the LNA component consists solely of guanine and adenine residues it is clear that these are also labeled by the Ulysis kit on the 5827 LNA, (FIG. 19). The N7 atoms of purine bases are likely to be involved in Hoogsteen base pairing, (74), which might well be interfered with by the addition of a large fluorescent label, however the molecular interaction needed for Watson-Crick base-pairing would be expected to the unaffected by the labelling. This is further supporting evidence that the major mechanism for LNA oligonucleotide binding to plasmid DNA is that of strand displacement by Watson-Crick base-pairing.

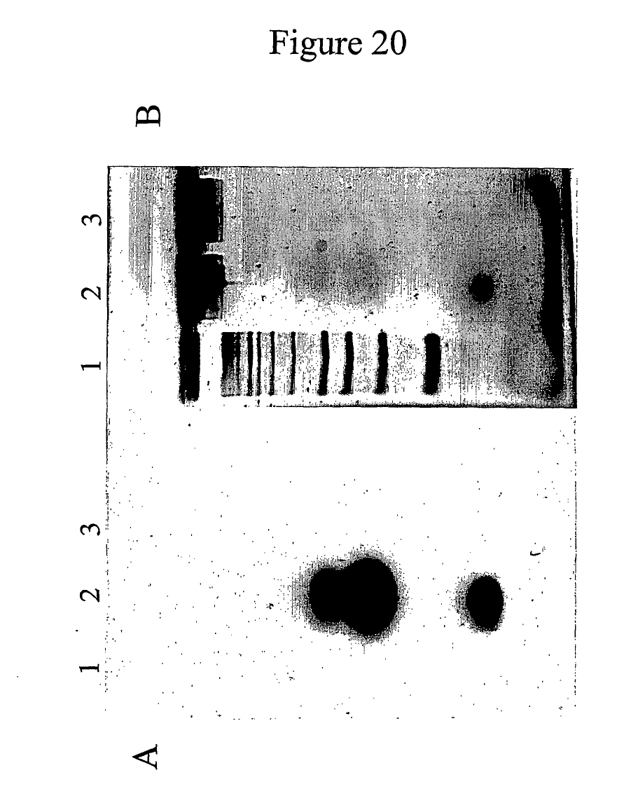

The binding of the PTOCpG or PTOGpC oligonucleotides to plasmid DNA under the conditions described above left some free oligonucleotide unbound to plasmid DNA, (FIG. 19). As the aim of these experiments was to analyse the effect of adding an immunostimulatory adjuvant, i.e. oligonucleotides with a CpG motif, (79), by binding to plasmid DNA via LNA oligonucleotides, it is clear that any free oligonucleotides that may demonstrate an adjuvant effect would have to be removed from plasmid preparations containing bound oligonucleotides. In order to confirm that the standard methodology for removing unbound oligonucleotides from preparations of plasmid plus bound oligonucleotides, i.e. separation through an S400HR spin column, (Amersham Pharmacia Biotech), a further experiment was performed. Oligonucleotide PTOCpG, labeled with Alexa Fluor 488, via the Ulysis labeling kit was bound to plasmid DNA, pGG2XEMPTY, as described above, except that prior to plasmid binding the oligonucleotide was heated for 10 mins. at 80° C. and then immediately plunged into ice. This was in order to disrupt any self-complementary interaction between the phosphorothioate bases within the oligonucleotide that might effect plasmid binding. The sample was split into two, with one half being separated through an S400HR spin column, both samples were then analysed by agarose gel electrophoresis, see FIG. 20. From FIG. 20, it can be clearly be seen that the large intensely labelled bands of free oligonucleotide present towards the middle and the bottom of the gel in the non S400HR treated sample have been completely removed in the S400HR treated sample.

FIG. 20 shows 2% agarose gel electrophoresis, in the absence of ethidium bromide, (EtBr), for analysis of supercoiled plasmid DNAs incubated with LNA oligonucleotides chemically labeled at the N⁷ G residue with a Ulysis Alexa Fluor 488 labelling kit, (Table 3, Molecular Probes, Leiden, Netherlands ).
(A) Gel analysis using the Labworks 4.0 package on the UVP EpiChemi Darkroom Bio Imaging System, 302 nm uv, Et Br filter (UVP, Cambridge, UK), before EtBr staining.
(B) As (A) after EtBr staining.
1) 1 ug of 1 kb DNA ladder, (Promega, Southampton, UK)
2) 2.5 ug pGG2xEMPTY plasmid incubated with Ulysis Alexa Fluor 488 labeled PTOCpG at 37° C.
3) 2.5 ug pGG2xEMPTY plasmid incubated with Ulysis Alexa Fluor 488 labeled PTOCpG at 37° C. and separated through a S400HR spin column.

Given the above data, similar binding experiments were performed to bind the unlabeled oligonucleotides PTOCpG or PTOGpC, respectively to plasmid gWiz, (endotoxin free), with any free oligonucleotide being removed from both samples by passing through an S400HR spin column as described, (data not shown). These plasmid samples were then suitable for use in RAW264.7 transfection experiments to look for murine tumour necrosis factor alpha, (TNFα), induction, see below.

RAW264. 7 Cell Culture, DNA Transfection and Oligonucleotide Incubation

The murine macrophage cell line RAW264.7 was maintained in RPMI 1640 medium with 10% FCS, 100 units/ml penicillin, 100 ug/ml streptomycin, 2 mM glutamine, (Life Technologies). RAW264.7 cells were grown to confluence in a 96-well plate (Lab tech, Nalge Nunc. Int.), washed once with 250 ul PBS, per well and incubated in 150 ul Optimem for two hours at 37° C. A transfection mixture of 0.01-10 uM CpG oligonucleotides +/− FuGENE6 Transfection Reagent (Roche Molecular Biochemicals, at a ratio of respectively 1 uM oligonucleotide: 0.5 ul FuGENE6), Optimem was added to a final volume of 100 ul, and the mixture was incubated at room temperature for 30 minutes. The transfection mixture was added to the RAW264.7 cells in Optimem and incubated for 14 hours at 37° C. As a control the same procedure was performed with solely FuGENE6 Transfection Reagent and 0.1-5 ug gWiz plasmid with and without transfection reagent, ratio of plasmid DNA 1 ug: 6 ul FuGENE6 transfection reagent

ELISA for Tumour Necrosis Factor Alpha, (TNFα), from RAW264. 7 Cells.

RAW264.7cells were grown and transfected with plasmids and oligonucleotides as described above in order to perform an ELISA assay based upon production of murine TNFα after stimulation with CpG motifs, (84, 85, 86, 87).

The culture supernatants were taken to detect murine TNFα levels using the Duoset ELISA development system kit, (R&D systems, Minneapolis), according to the manufacturer's protocol, after 14 hours incubation as described above. Then fresh media was added to the RAW267.4 cells for harvesting at 24 hours post transfection to perform a luciferase assay upon the lysed cells, see below for details. After dilution of supernatant samples in Reagent diluent, (1% BSA in PBS), the ELISA was performed in 96 well Nunc Immuno ELISA plates, (Nalge Nunc), and the absorbance was measured at 450 nm on a Molecular Devices Spectra Max 190 and the murine TNFα values were calculated using a 4-PL curve fit on the Softmax Pro 3.1.2 software. Results were expressed as mean of duplicate samples (ng/ml).

Calculation of Dose Equivalents of CpG Oligonucleotides that Could Theoretically be Bound to Plasmid gWiz by CpG ‘Hybrid’ PTO/LNA Oligonucleotides.

The doses of plasmid gWiz, (ug), that were transfected into RAW264.7 cells were converted into equivalent doses of CpG oligonucleotide, (uM), that could be theoretically bound to gWiz plasmid if the CpG oligonucleotide was attached to an LNA oligonucleotide with binding sites in the gWiz plasmid, ie. as PTOCpG, Table 3. The calculation assumes that 1 ug of a 5 kb plasmid is about 0.3 pmoles, (GTS ‘PNA clamp’, manufacturer's instructions), plasmid gWiz is 6.7 kb and therefore 1 ug is about 0.23 pmoles. Theoretically, 1 ug of plasmid can bind 0.23 pmol of PTOCpG oligonucleotide per binding site, and 6 binding sites are present on the gWiz plasmid, (Table 3). Therefore, when all six binding sites on plasmid gWiz are occupied or six PTOCpG oligonucleotides have bound by Watson-Crick basepairing of the LNA, 6 times 0.23=1.2 pmol CpG oligonucleotide is bound. The total volume of the ELISA assay is 250 ul, which results in a concentration of 4.8 uM CpG. In conclusion, 1 ug of plasmid is comparable to 4.8 uM of CpG oligonucleotide and this comparison has been used to normalise murine TNFα levels between gWiz plasmid and free CpG oligonucleotides in RAW264.7 transfections.

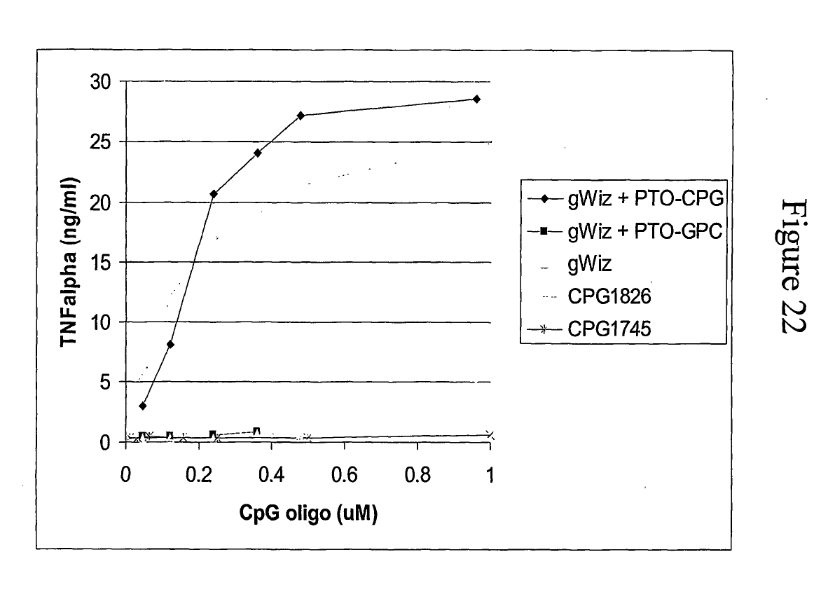

An example of this, as a means of predicting whether the RAW264.7 transfection assay, with gWiz plasmid and theoretical numbers of potentially bound CpG oligonucleotides, to generate TNFα signal would be sensitive to distinguish between the presence and absence of plasmid bound adjuvant is shown in FIG. 22.

FIG. 22 shows the dose response curve of the adjuvant effect induced by lipid based transfection of PTOCpG, bound to gWiz plasmid, into RAW264.7 as TNFα levels, compared to gWiz plasmid alone, CPG1826 oligonucleotide and negative controls CPG1745 oligonucleotide and gWiz bound to PTOGpC.

This clearly demonstrates that the CPG1826 oligonucleotide shows a CpG adjuvant effect whereas the CPG1745 negative control does not. The conversion of plasmid dose to CpG dose also strongly suggests that if the expected six CpG oligonucleotides, (i.e. PTOCpG, Table 3), were bound to gWiz plasmid DNA, a 12-30 fold increase in TNFα signal, over gWiz plasmid DNA would be expected. Note that gWiz contains one murine 6mer ‘core’ CpG sequence that could be largely responsible for the baseline TNFα production in RAW264.7 transfected with this plasmid, (83).

Demonstration of CpG ‘Hybrid’ PTO/LNA Oligonucletides Adjuvant Effect in RAW264. 7 Cells

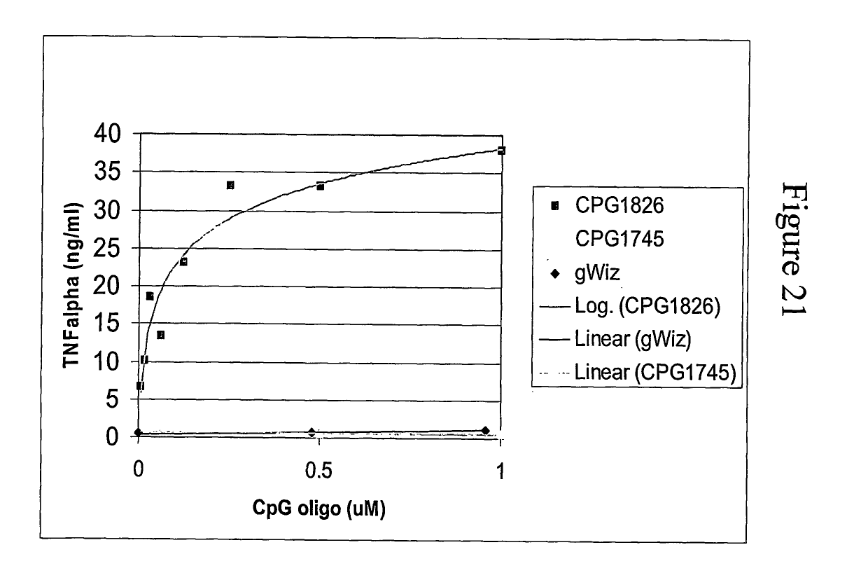

Data demonstrating the adjuvant effect of having oligonucleotide PTOCpG bound to plasmid gWiz, as TNFα production, after transfection into RAW264.7 cells is shown in FIG. 22. FIG. 22 also compares plasmid doses with that of CpG oligonucleotides from a dose range of 1 μM of CPG1826 and CPG1745, serially diluted two-fold, down to 0.078 μM. The conversion of plasmid gWiz, (μg), into CpG (μM), was performed as described above and as as shown in FIG. 21.

FIG. 21 shows a dose response curve of the adjuvant effect of CpG oligonucleotides, compared to basal levels induced by transfection of plasmid gWiz, when incubated with RAW264.7 cells. This is displayed graphically as oligonucleotide concentration in μM against TNFα production in ng/ml. The log, or linear refers to the trendline connecting the points.

The graph plotted in FIG. 22 was used to determine the difference in levels of TNFα induced by gWiz plasmid plus and minus bound PTOCpG and free oligonucleotide CPG18216. If plasmid gWiz has PTOCpG oligonucleotide bound, there is a 7 to 27 fold increase in TNFα levels over that induced by plasmid alone. This compares well with both that predicted from FIG. 21, (i.e. 12 to 30 fold), and the differential between free CPG1826 and gWiz plasmid induced TNFα levels, 5 to 22 fold in this experiment, FIG. 22. This data clearly demonstrate that an immune adjuvant, i.e. CpG oligonucleotide, can be added to plasmid DNA coupled by LNA oligonucleotides. It is thought that this assay is based upon uptake of either transfected plasmid DNA or free oligonucleotides into the endosomes of RAW264.7 cells where CpG motifs are thought to interact with the TLR9 receptor to cause the induction of TNFα production, (82, 83). Transfection of plasmid with FuGENE6 into RAW264.7 cells has been previously shown to result in most of the plasmid DNA being taken up into the endosome, (data not shown).

Luciferase Assay from RAW264.7 Cells.

To measure transfection efficiency, 24 hours after transfection, cells were washed once with PBS and lysed by addition of 100 ul per well of low detergent Magnus Lysis Buffer, (10 mM EDTA 0.25%, TritonX-100, 10 mM DDT, 250 mM Hepes pH7.5). In a 96 well black and white isoplate (Wallac, Perkin Elmer), 501 μl of the lysate was assayed together with 200 μl of luciferase assay reagent (Promega). Luciferase activity, (relative light units, RLU), was measured as counts per minute on the Victor² 1420 multilabel HTS counter on the luminescence program, (Wallac, Perkin Elmer). Total protein concentration was calculated by Coomassie Plus protein assay reagent kit, (Perbio), according to the manufacturer's protocol. In a clear 96 well flat-bottomed plate (Costar), 5 μl cell lysate, 145 μl of water (Sigma) and 150 μl of coomassie blue reagent were mixed and the absorbance was measured at 595 nm on a Molecular Devices Spectra Max 190. Results were expressed as mean of duplicate samples (μg/ml). Luciferase activity was expressed as relative light units (RLU)/mg of total protein.

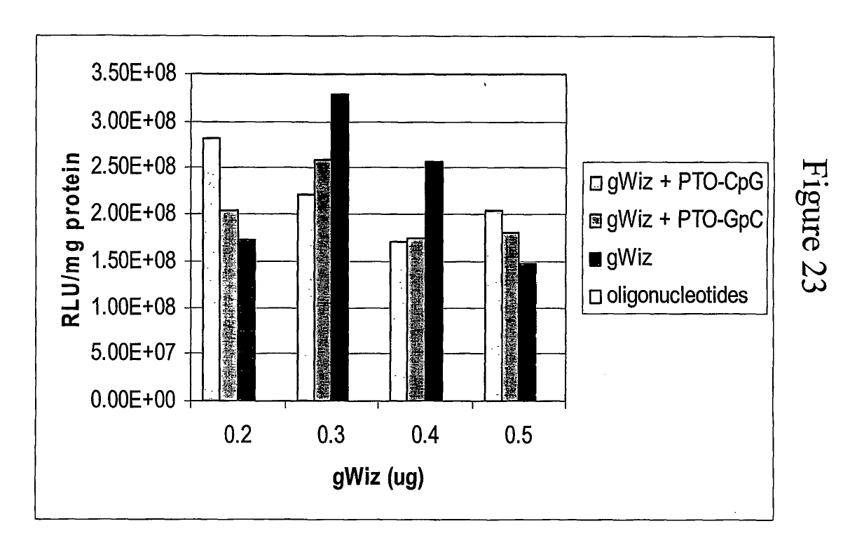

An example of this is shown in FIG. 23, where the luciferase activity derived from four different doses of either gWiz plasmid alone, (in μg), or with bound PTOCpG or bound PTOGpC 1745 are compared.

FIG. 23 shows the luciferase expression in RAW264.7 cells derived from lipid-based transfection of gWiz plasmid, with and without bound oligonucleotides, as relative luminescence units (RLU) per mg of protein. The sample labelled oligonucleotides represents a negative control of simply oligonucleotide incubation with RAW264.7 cells.

The data is taken directly from the cell samples used to generate the ELISA results plotted in FIG. 22, and clearly shows that the presence of bound CpG oligonucleotide on gWiz plasmid DNA does not reduce expression of luciferase. Given also the adjuvant effect of the bound CpG oligonucleotides, this demonstrates that using LNA oligonucleotides to bind an immune adjuvant to plasmid DNA expressing an encoded antigen can lead to both an immune adjuvant effect and high level antigen expression.

### EXAMPLE 10

### Methods for Attaching Functional Peptides as Conjugates to LNA Oligonucleotides for Binding to Plasmid DNA and Improving Gene Transfer Efficiency

Use of Heterobifunctional Crosslinkers to Attach Modified Peptides to LNA Oligonucleotides.

The use of various technologies to achieve the coupling and/or co-synthesis of peptide oligonucleotide conjugates has been reviewed recently, (88). One of the ways of achieving the formation of peptide to oligonucleotide conjugates is to synthesize each with a modified group, such as a peptide with a C-terminal cystine residue to introduce a free sulphydryl group and an oligonucleotide, such as an LNA oligonucleotide, with a 5′ modification to introduce a free primary amino group. Such molecules could then be coupled by the use of the wide range of heterobifunctional linkers that are commercially available, (Perbio Catalogue 2002, Perbio), eg. N-succinmidyl-3-(2-pyridyldithio)propionate (SPDP, Perbio, 89), maleimide-based such as: 4-(maleimidomethyl)-1-cyclohexane-carboxylic acid N-hydroxysuccinimide ester (SMCC, Perbio, 90), Sulfo-MBS m-Maleimidobenzoyl-N-hydroxysulfosuccinimide ester, (Sulfo-MBS, Perbio), N-(6-Maleimidocaproyloxy) sulfosuccinimide, (Sulfo-EMCS, Dojindo Laboratories, Kumamoto, Japan). Functional peptides that could be coupled to LNA include those containing nuclear localisation signals, (nls), see Table 2, (19), or membrane transport sequences, (MTS, 26) and other functional peptides as described earlier in this work. The LNA oligonucleotides that could be coupled to peptides in this fashioned are similar to those listed in Table 3. These could be coupled to one another either prior to binding of the peptide: LNA oligonucleotide to plasmid DNA or the coupling conjugations could be performed on the modified LNA oligonucleotide whilst it is bound to plasmid DNA in a similar manner to that described for PNA oligonucleotides, (Example 4).

Use of Disulphide Crosslinking of Peptides to LNA Oligonucleotides.

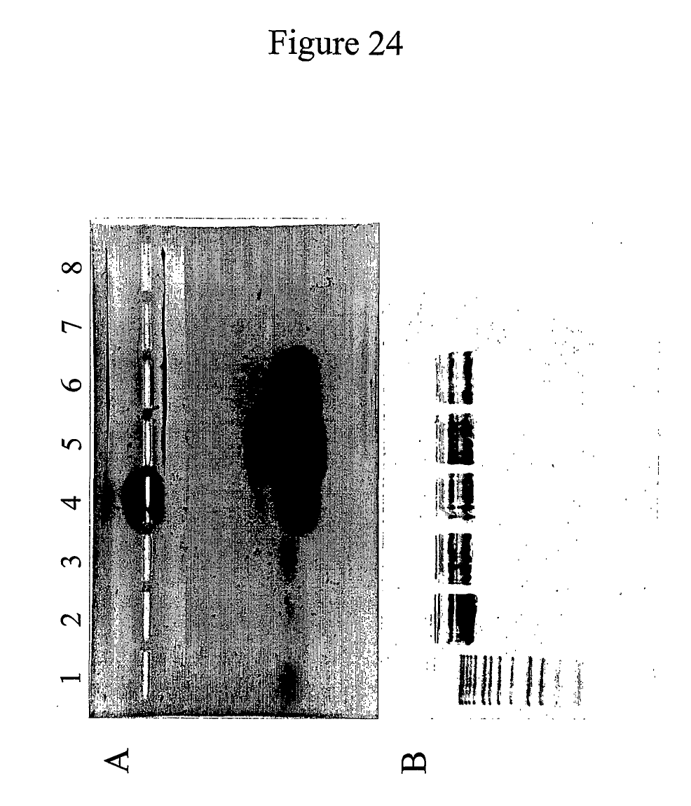

A further way to achieve the conjugation of functional peptides to oligonucleotides is to synthesize both molecules with either free sulphydryl groups or cleavable disulphide modified groups to allow coupling by the formation of disulphide bonds. Peptides were synthesized as described above with a C-terminal cystine residue and examples include the peptides listed in Table 3, which have been described previously in Example 4, apart from FGF, (94), which was synthesized by Genemed Synthesis Inc., California, USA. A 100% LNA oligonucleotide with a 5′ C6 S-S modification incorporated, 5′SHGA, was synthesized by Proligo LLC, Boulder, USA, Table 3. Peptides were resuspended at 1-2 mg/ml in PBS and labelled with Alexa Fluor 568 dye, as described previously, Example 4. A twenty-fold molar excess of each of the four fluorescently labelled peptides listed in Table 2, respectively, was mixed with 1000 pmoles of 5′ SHGA LNA oligonucleotide, (which had already bound to gWiz plasmid DNA as described previously). This was incubated for 1 hour at room temperature with immobilised TCEP disulphide reducing gel, (Perbio), to break disulphide bonds. The mixture was then removed from the gel by centrifuging, 2 minutes, 3000 rpm in an Eppendorf microfuge and the reduced peptide/oligonucleotide/plasmid mixture was incubated at 4° C. for 16 hours with vigorous shaking to enable the peptide-oligonucleotide conjugates to form. The samples were then passed through an S400HR spin column to remove any free unbound peptides or oligonucleotides from the plasmid DNA. 2.5 ug samples of the plasmid DNA, with bound LNA oligonucleotides coupled to fluorescent peptides, were then visualised on an agarose gel, FIG. 24.

FIG. 24 shows 2% agarose gel electrophoresis, in the absence of ethidium bromide, (EtBr), for analysis of supercoiled plasmid DNAs incubated with Alexa Fluor 568 labeled peptides conjugated to the 5′ SHGA LNA oligonucleotide, (Table 3), and then separated from free unbound LNA oligonucleotide by gel exclusion chromatography through Microspin S400HR columns, (Amersham Pharmacia Biotech, Little Chalfont, UK). Peptide sequences are listed in Table 2.
(A)) Gel analysis using the Labworks 4.0 package on the UVP EpiChemi Darkroom Bio Imaging System, 302 nm uv, Et Br filter (UVP, Cambridge, UK), before EtBr staining.
(B) As (A) after EtBr staining.
1) 1 ug of 1 kb DNA ladder, (Promega, Southampton, UK)
2) 2.5 ug gWiz plasmid.
3) 2.5 ug gWiz plasmid incubated with SV40nls peptide/5′SHGA LNA oligonucleotide at 37° C.
4) 2.5 ug gWiz plasmid incubated with AdF peptide/5′SHGA LNA oligonucleotide at 37° C.
5) 2.5 ug gWiz plasmid incubated with M9 peptide/5′SHGA LNA oligonucleotide at 37° C.
6) 2.5 ug gWiz plasmid incubated with FGF peptide/5′SHGA LNA oligonucleotide at 37° C.
7) Empty
8) Empty

Plasmid bands, with fluorescently bound peptide LNA conjugates, could be detected and quantified, (data not shown), for all four peptides investigated, FIG. 24, but not for plasmid gWiz alone. Plasmid gWiz incubated with similarly treated peptides did not show fluorescent bands upon agarose gel electrophoresis, although the sv40nls peptide, Table 2, has previously been shown to bind plasmid DNA alone, (data not shown). This data suggests that peptide: LNA oligonucleotides can be formed in this manner and still show binding to plasmid to add functionality to plasmid DNA. For example, plasmids with nls peptides attached via LNA could be transfected into mammalian cells and any effects upon increasing gene expression monitored as described in Example 4. Membrane transport sequence peptides, such as FGF, (Table 2, 94) coupled to LNA oligonucleotides and bound to plasmid could be analysed for their effect upon increased uptake of fluorescently labelled plasmid DNA into mammalian cells by confocal microscopy in a similar manner to that described in Example 8. Similar assays could be performed for plasmids bound with LNA oligonucleotides conjugated to peptides with a dual function such as melittin, (77, 95), which shows enhanced endosomal release and nuclear uptake properties which can be conferred to plasmid DNA, (95).

Use of “Native Ligation” to Conjugate Peptides to LNA Oligonucleotides

A further method that could be used to conjugate functional peptides to LNA oligonucleotides for binding to plasmid DNA is described as ‘native ligation’, (91, 92) and is commercially available from Link Technologies, Strathclyde, Scotland. The method requires the synthesis of an N-terminal thioester functionalised peptide and a 5′ cysteinyl LNA oligonucleotide. Functionalised peptides and oligonucleotides can be conjugated directly post synthesis without further purification using TCEP in aqueous solution, (91). Peptide-LNA oligonucleotide conjugates formed in this and other ways can be purified by reverse phase HPLC, (88, 91), prior to binding to plasmid DNA.

Use of Solid Phase Co-synthesis Methods for Formation of LNA Oligonucleotide: Peptide Conjugates

A still further method that could be used to generate conjugates of functional peptides and LNA oligonucleotides for binding to plasmid DNA is based upon a solid phase method that allows co-synthesis of 3′ peptide conjugates of oligonucleotides, (93). The method is based upon a homoserine-functionalized solid support system that allows both oligonucleotide and peptide assembly under standard conditions, (93).

### OTHER PUBLICATIONS

1) Rajur et al., 8(6):935-940 (1997).
2) Brinkley, M., 3(1):2-13 (1992).
3) Mattaj, I & Engelmeier, L., Annual Review Biochemistry 67:265-306 (1998)
4) Gorlich, D. & Kutay, U., Annual Review Cellular & Developmental Biology 15:607-660 (1999).
5) Zanta et al., Proc. Natl. Acad. Sci. USA 96: 91-96 (1999).
6) Sebestyen et al., Nature Biotech. 16: 80-85 (1998).
7) Branden et al., Nature 17: 784787 (1999).
8) Zhang et al., Gene Therapy 6: 171-181 (1999).
9) Subramanian et al., Nature 17: 873-877 (1999).
10) Chang et al., Virology 189: 821-827 (1992).
11) Vives et al., Journal Biological Chemistry 272 (25): 16010-16017 (1997).
12) Derossi et al., Journal Biological Chemistry 269 (14):10444-10450 (1994).
13)Derossi et al., Trends in Cell Biology 8: 84-87 (1998).
14) Prochanitz, A., Current Opinion in Neurobiology 6: 629-634 (1996).
15) Prochiantz, A. & Theodore, L., BioEssays 17(1): 39-44 (1995).
16) Eguchi et al., J. Biol Chem 272: 17640-17647 (1997).
17) Burglin, T. & De Robertis, E., EMBO J. 6: 2617-2625 (1987).
18) Ballas, N. & Citovsky, V., Proc. Natl. Acad. Sci. USA 94: 10723-10728 (1997).
19) Nigg, E., Nature 386: 779-787 (1997).
20) Blanke et al., Proc. Natl. Acad. Sci. USA 93: 8437-8442 (1996).
21) Morris et al., Nucleic Acids Research 27 3510-3517 (1999).
22) Schmolke et al., Journal of Virology 69: 1071-1078 (1995).
23) Wychowski et al., EMBO J. 5: 2569-2576.
24) Xia et al., Journal of Virology 66: 914-921 (1992).
25) Dean, D., Adv. Drug Delivery reviews 44: 81-95 (2000).
26) Lindgren et al., Trends in Pharmaceutical Sciences 21: 99-103 (2000).
27) Elliot, G. & O' Hare, P, Cell 88: 223-233 (1997).
28) Kawana et al., Journal of Virology 75 (5): 2331-2336 (2001).
29) Pastan, I & Fitzgerald, D., Science 254: 1173-1177 (1991).
30) Oess, S. & Hildt, E., Gene Therapy 7: 750-758 (2000).
31) Mechtler, K. & Wagner, E, New Journal of Chemistry 21: 105-111 (1997).
32) Pillot et al., Journal Biological Chemistry 271: 28757-28765 (1996).
33) Subbarao et al., Biochemistry 26 (11): 2964-2972 (1987).
34) Niidome et al., Journal Biological Chemistry, 272: 15307-15312 (1997).
35) Wyman et al., Biochemistry 36: 3008-3017 (1997).
36) Vogel et al., Journal American Chemical Society 118: 1581-1586 (1996).
37) Kichler et al., Bioconjugate Chemistry 8: 213-221 (1997).
38) Puls, R. & Minchin,.M., Gene Therapy 6: 1774-1778 (1999).
39) Goldman et al., Cancer Research 57: 1447-1451 (1997)
40) Kircheis et al., Gene Therapy 4: 409-418 (1997)
41) Kawakami et al., Gene Therapy 7: 292-299 (2000).
42) Fajac et al., Human Gene Therapy 10: 395-406 (1999).
43) Zanta et al., Bioconjugate Chemistry 8: 839-844 (1997).
44) Hung et al., Cancer Research 61: 1080-1088 (2001).
45) Hart et al., Gene Therapy, 2: 552-554 (1995).
46) Deonarain, M., Expert Opinion on Therapeutic Patents 8(1): 53-69 (1998).
47) Cristiano, R. & Roth, J., Journal Molecular Medicine 73: 479-486 (1995)
48) Uherek, C. et al., Journal of Biological Chemistry, 273: 8835-8841 (1998).
49) Chattergoon et al., Nature Biotechnology 18: 974-979 (2000).
50) Zelphati et al., Human Gene Therapy 10: 15-24 (1999).
51) Maniatis et al., ‘Molecular Cloning: a laboratory manual’ 2^{nd} Edition, (1992)
52) Egholm et al., Nucleic Acids Research 23: 217-222 (1995).
53) Zhang et al., Nucleic Acids Research 28 (17): 3332-3338 (2000).
54) Griffith et al., Journal American Chemical Society 117: 831-832, (1995).
55) Nielsen, P. & Eghohn, M. ‘An introduction to PNA’ Chapter 1 In Nielsen, P. & Eghohn, M (Eds.) Peptide Nucleic Acids: Protocols and Applications (1999).
56) Butz, E. & Bevan, M., Journal of Immunology 160: 2139-2144, (1998).
57) Tuting, et al., Journal of Immunology 160: 1139-1147, (1998).
58) Harrison, et al., Vaccine 19: 1820-1826, (2001).
59) Vasilakos, et al., Cellular immunology 204: 64-74 (2000).
60) Rhodes, J. et al., Nature 377: 71-75 (1995).
61) Gherardi, M. et al., Journal of Virology 74 (14): 6278-6286.
62) Alderton, W. et al., Biochemical Journal 357: 593-615, (2001).
63) Scheerlinck, JP. ‘Genetic adjuvants for DNA vaccines’, Vaccine 19: 2647-2656, (2001).
64) Reyes et al., Vaccine 19: 3778-3786, (2001).
65) Sato, Y. et al., Science 273 (5273): 352-354 (1996).
66) Hemmi, H. et al., Nature 408: 740-745, (2000)
67) Beutler, B., Current Opinion in Microbiology 3: 23-30 (2000).
68) Thoma-Uszynski, S. et al., Journal of Immunology 165: 3804-3810 (2000).
69) Mitchell, P. & Tijan, R., Science 245: 371-378, (1989).
70) Courey, A. & Tijan, R., Cell 55: 887-898, (1988).
71) Tanaka et al., Molecular and Cellular Biology 14: 6046-6055, (1994).
72) Ma, J. & Ptashne, M., Cell, 51: 113-119, (1987).
73) Seipel et al., EMBO Journal 11: 4961-4968, (1992).
74) Obika et al., Tetrahedron Letters 41: 221-224, (2000).
75) Torigue et al., Journal of Biological Chemistry 276(4): 2354-2360, (2001).
76) Smulevitch et al., Nature Biotechnology 14: 1700-1704, (1996).
77) Kichler et al., Bioconjugate Chemistry 8: 213-221, (1997).
78) Bukanov et al., Proc. Natl. Acad. Sci. USA 95: 5516-5518, (1998).
79) Klinman et al., Vaccine 17: 19-25, (1999).
80) Weeratna et al., Vaccine 18: 1755-1762, (2000).
81) Shirota et al., Journal of Immunology 167: 66-74, (2001).
82) Hacker et al., EMBO Journal 17: 6230-6240, (1998).
83) Bauer et al., Proc. Natl. Acad. Sci. USA 98 (16): 9237-9242, (2001); International patent application: ‘Process for high throughput screening of CpG-based immuno-agonist/antagonists’, WO02/228009A2, (2002); S. Bauer, personal communication, (unpublished observation).
84) Jin et al., Journal of Immunology 165: 5153-5160, (2000).
85) Dalpke et al., Journal of Immunology 166: 7082-7089, (2001).
86) Crabtree et al., Infection and Immunity 69(4): 2123-2129, (2001).
87) Gao et al., Journal of Immunology 166: 6855-6860, (2001).
88) Tung, C. H. and Stein, S., Bioconjugate Chemistry 11(5): 605-618, (2000).
89) Wang et al., Bioconjugate Chemistry 8: 878-884, (1997).
90) Harrison, J. and Balasubramanian, S., Nucleic Acids Research 26 (13): 3136-3145, (1998).
91) Stetsenko, D. and Gait, M., J. Org. Chem. 65: 49004908, (2000).
92) Stetsenko, D. and Gait, M., Nucleosides, Nucleotides & Nucleic Acids 19(10-12): 1751-1764, (2000).
93) Stetsenko, D. and Gait, M., Bioconjugate Chemistry 12: 576-586, (2001).
94) Rojas et al., Nature Biotechnology 16: 370-375, (1998).
95) Ogris et al., Jourmal of Biochemistry 276 (50): 47550-47555, (2001).

### ABBREVIATIONS

LNA—Locked Nucleic Acid.
NLS—Nuclear Localisation Signal.
HnRNP—Heterogeneous nuclear ribonucleoprotein.
HIV-1—Human immunodeficiency virus -1
CMV—Cytomegalovirus
HSV—Herpes Simplex Virus
HPV—Human Papillomavirus
PEI—Polyethylenimine
TAMRA—Carboxytetramethylrhodamine
ROX—Carboxy-X-rhodamine
PNA—Peptide nucleic acid
GFP—Green fluorescent protein
PMID—Particle mediated immunotherapeutic delivery
TAE—Tris Acetate EDTA, pH 8.0
DMRIE-C—a 1:1, (M/M) mix of 1,2-dimyristyloxypropyl-3-dimethyl-hydroxyl ethyl ammonium bromide and cholesterol

1. A locked nucleic acid conjugate comprising an oligonucleotide comprising at least one locked nucleic acid base and a functional moiety.

2. A locked nucleic acid conjugate as claimed in claim 1, wherein the functional moiety is a member selected from the group consisting of: Fluorescent labels; nuclear localisation peptides; peptides that have the ability to cross the plasma membrane of eukaryotic cells (“cell penetrating peptides”); endosomal escape peptides; cell targeting and binding peptides or protein; peptides or proteins with transcription activation domains; and molecules having adjuvant or immunostimulatory activity.

3. A locked nucleic acid conjugate as claimed in claim 1 wherein the oligonucleotide is between 7 to 25 bases in length.

4. A locked nucleic acid conjugate as claimed in claim 1 wherein at least 50% of the bases are locked nucleic acid bases.

5. A locked nucleic acid conjugate as claimed in claim 1 wherein the at least one locked nucleic acid base is a member selected from the group consisting of O—, (oxy) S—, (thio) ad NH—₂ (amino) bridged locked nucleic acid base.

6. A locked nucleic acid conjugate as claimed in claim 1 wherein the oligonucleotide is free from self-complementary base pairings.

7. A locked nucleic acid conjugate as claimed in claim 1 comprising a cleavable linkage between the functional moiety and the oligonucleotide which is selectively cleavable after administration to a patient.

8. A locked nucleic acid conjugate as claimed in claim 1 wherein the functional moiety is an immunostimulatory oligonucleotide containing at least one unmethylated CG di-nucleotide motif.

9. A complex comprising a locked nucleic acid conjugate as claimed in claim 1, and a DNA sequence having a complementary sequence to the oligonucleotide, and encoding a gene under the control of a promoter.

10. A complex as claimed in claim 9 wherein at least one further locked nucleic acid conjugate is present which is bound to a complementary sequence within the locked nucleic acid complex which is itself bound to the DNA sequence.

11. A complex as claimed in claim 10 comprising an array of locked nucleic acid conjugates bound to a single locked nucleic acid complementary sequence within the DNA, formed by locked nucleic acid: locked nucleic acid hybridisation between locked nucleic acid oligonucleotide.

12. A complex as claimed in claim 9 wherein the gene encodes for a therapeutic protein or an antigen.

13. A complex as claimed in claim 10 wherein a plurality of locked nucleic acid complexes are bound to a plurality of complementary sequences within said DNA sequence.

14. A complex of claim 12 wherein the antigen is capable of raising an immune response against a pathogen or a tumour.

15. A complex as claimed in claim 9 wherein the DNA sequence is in the form of an open circular or supercoiled plasmid.

16. A pharmaceutical composition comprising a complex as claimed in claim 9 and a pharmaceutically acceptable carrier or dilulent.

17. A pharmaceutical composition as claimed in claim 16 wherein the complex is coated on to a microprojectile.

18. A pharmaceutical composition as claimed in claim 17 wherein the microprojectiles are gold beads.

19. A device loaded with the pharmaceutical composition of claim 16.

20-23. cancelled.

24. A process for the the preparation of a pharmaceutical composition comprising the step of hybridising the locked nucleic acid conjugate of claim 1 with a plasmid capable of expressing a gene encoding an antigen or therapeutic protein, and formulating the resulting complex with a pharmaceutical acceptable carrier.

25. An oligonucleotide comprising a first region comprising an oligonucleotide sequence having at least one locked nucleic acid, and a second region comprising an immunostimulatory oligonucleotide region containing at least one unmethylated CG di-nucleotide motif.

26. An oligonucleotide as claimed in claim 25 wherein the first locked nucleic acid containing region and the second immunostimulatory oligonucleotide region are separated by a phosphoramidate region, and wherein the second immunostimulatory oligonucleotide region comprises a phosphorothioate backbone.
